(19) United States
(12) Patent Application Publication
(10) Pub. No.: US 2022/0193433 A1
(43) Pub. Date: Jun. 23, 2022

(54) DETECTING AND ADDRESSING IRREGULAR MOTION TO IMPROVE DEFIBRILLATION SHOCK RECOMMENDATIONS

(71) Applicant: Stryker Corporation, Kalamazoo, MI (US)

(72) Inventors: Fred W. Chapman, Newcastle, WA (US); Ryan William Apperson, Bothell, WA (US); Steven Barry Duke, Bothell, WA (US); Michelle Liu, Redmond, WA (US); Thangeswaran Natarajan, Bothell, WA (US); Daniel W. Piraino, Seattle, WA (US); Tyson G. Taylor, Bothell, WA (US); Robert G. Walker, Seattle, WA (US)

(21) Appl. No.: 17/559,795

(22) Filed: Dec. 22, 2021

Related U.S. Application Data
(60) Provisional application No. 63/130,143, filed on Dec. 23, 2020.

(51) Int. Cl. A61N 1/39; G16H 20/30; G16H 40/63

(52) U.S. Cl. CPC A61N 1/3987; A61N 1/39044; A61N 1/3993; G16H 20/30; G16H 40/63; A61N 1/3925

(57) ABSTRACT

Systems, devices, and methods for detecting and addressing irregular motion to improve defibrillation shock recommendations are described. In an example method performed by a medical device, an electrocardiogram (ECG) of an individual receiving chest compressions is detected. In addition, irregular motion of the individual is detected. If a magnitude of the irregular motion is greater than or equal to a threshold, a remedial action is performed. In some examples, the medical device refrains from generating a recommendation indicating whether the ECG includes a shockable rhythm and/or whether a defibrillation shock is recommended. In some instances, the medical device outputs the recommendation with a certainty of the recommendation. In some cases, the medical device outputs a warning and generates the recommendation in response to receiving an input signal indicating a manual override.

### CROSS-REFERENCE TO RELATED APPLICATION

This application claims the priority of U.S. Provisional Application No. 63/130,143, titled “DETECTING AND ADDRESSING IRREGULAR MOTION TO IMPROVE DEFIBRILLATION SHOCK RECOMMENDATIONS,” which was filed on Dec. 23, 2020 and is incorporated by reference herein in its entirety.

### BACKGROUND

Cardiac arrest is a condition in which an individual's heart ceases to function effectively. During cardiac arrest, the brain and other vital organs are unable to receive sufficient oxygenated blood, which can result in a sudden loss of consciousness. If untreated shortly after onset, cardiac arrest can result in long-term deficits or death. Thus, effective treatments must be applicable in a variety of environments where cardiac arrest is likely to occur, such as environments outside of hospitals or other specialized facilities for administering medical care.

Cardiopulmonary resuscitation (CPR) is a treatment that forces blood to vital organs using chest compressions, which can be administered manually or via a chest compression device, such as the LUCAS 3®, by Stryker Corporation of Kalamazoo, Mich. CPR is indicated for individuals experiencing cardiac arrest and can slow down damage to the vital organs by providing at least some blood flow despite the heart's disfunction. However, the underlying cause of the cardiac arrest is not treatable by CPR.

Some forms of cardiac arrest are the result of abnormal heart rhythms, such as ventricular fibrillation (VF) and pulseless ventricular tachycardia (V-tach). VF and pulseless V-tach are treatable by defibrillation, which is the delivery of an electrical shock to the heart. Because a defibrillation shock can be dangerous if administered to individuals without VF or pulseless V-tach, a medical device will generally identify and/or assist in the diagnosis of VF and pulseless V-tach based on electrocardiograms (ECGs). An ECG includes one or more lead signals that are indicative of the electrical activity of an individual's heart over time.

### BRIEF DESCRIPTION OF THE DRAWINGS

FIG. 1 illustrates an example of an emergency environment in which a rescuer is assisting a patient in cardiac arrest.

FIG. 2 illustrates example signaling associated with accommodating for irregular motion of a patient as a monitor-defibrillator is operating in manual mode.

FIG. 3 illustrates an example of a defibrillator visually displaying ECG-related data.

FIG. 4 illustrates an example of data stored at least temporarily in memory of a defibrillator.

FIG. 5 illustrates an example process for selectively outputting an accuracy of a shock recommendation based on irregular motion detection.

FIG. 6 illustrates an example process for selectively pausing an advisory mode of a defibrillator based on irregular motion detection.

FIG. 7 illustrates an example process for selectively allowing a user to manually override pausing of an advisory mode of a defibrillator based on irregular motion detection.

FIG. 8 illustrates an example process for identifying a shockable rhythm in ECG data that includes a chest compression artifact.

FIG. 9 illustrates an example of an external defibrillator configured to perform various functions described herein.

### DETAILED DESCRIPTION

The current disclosure describes detecting and addressing irregular motion to improve defibrillation shock recommendations. Various implementations described herein relate to systems, methods, and devices for selectively analyzing an ECG signal with chest compression artifact based on irregular motion detection. In some cases, in a user-selectable analysis mode, a medical device determines whether a shockable rhythm (e.g., VF or pulseless V-Tach) is present in an ECG signal of an individual that is obtained when the individual is receiving chest compressions. In addition, the medical device determines whether an irregular motion of the individual is greater than a particular threshold. The irregular motion is different than (e.g., independent of) motion caused by the chest compressions and, for example, relates to motion associated with transporting the individual or other non-periodic movements. If the medical device determines that the irregular motion is less than the particular threshold, the medical device outputs a recommendation based on whether the shockable rhythm is present. In examples in which the medical device determines that the irregular motion is greater than or equal to the particular threshold, the medical device refrains from outputting the recommendation, outputs a certainty (or confidence level) of the recommendation, or refrains from outputting the recommendation unless the medical device receives a manual override. Various techniques described herein are applicable to monitor-defibrillators operating in manual mode.

In some implementations the medical device is configured to determine whether the shockable rhythm is present in an ECG signal of an individual that is obtained when the individual is not receiving chest compressions. Thus, in these implementations, the chest compression artifact is missing from the ECG signal. Nevertheless, the medical device refrains from outputting a recommendation, outputs a certainty of the recommendation, or refrains from outputting the recommendation unless a user inputs a manual override, when the irregular motion of the individual is greater than the particular threshold.

Various implementations described herein address specific problems in the technical field of medical devices. When a patient experiences cardiac arrest, chest compressions can preserve the function of the patient's brain and vital organs until the patient's heart restores spontaneous blood circulation. If the cardiac arrest is the result of a shockable arrhythmia, however, the patient's heart may not restore spontaneous blood circulation until the arrhythmia is treated with defibrillation, and often until after CPR is administered for a period of time (seconds to minutes). In general, a defibrillator determines whether the patient is experiencing a shockable arrhythmia based on the ECG of the patient. The ECG is obtained by measuring electrical signals output by the patient's heart via electrodes attached to the patient's chest. The ECG is sensitive to jostling and changes in the impedance of the patient's chest as well as quality of electrode to skin contact, skin stretching, electrostatic interference, etc. Chest compressions are a major source of noise in the ECG.

Traditionally, chest compressions were temporarily paused in order to obtain a sufficient quality of ECG to determine whether the patient had a shockable arrhythmia. Any pause in chest compressions, however, would temporarily prevent the patient's brain and vital organs from receiving blood, which would potentially result in long-term damage. Thus, it is advantageous to reduce pauses in chest compressions.

To accurately determine whether the patient has a shockable heart rhythm while minimizing pauses in chest compressions, various signal processing techniques for reducing chest compression artifact in ECG signals have been developed. These techniques include various techniques for identifying and/or removing the chest compression artifact. In various examples, an Automated External Defibrillator (AED) is able to accurately identify the presence or absence of a shockable rhythm in an ECG signal from which the chest compression artifact has been removed. Thus, the AED determines whether a defibrillation shock is indicated while chest compressions are administered to the patient. The AED, in turn, automatically charges and administers the defibrillation shock.

In various implementations described herein, these signal processing techniques are adapted for use by monitor-defibrillators, rather than AEDs. In some cases, a monitor-defibrillator operating in manual mode activates chest compression artifact filtering based on a selection by a user. When the filtering is activated, the monitor-defibrillator removes the chest compression artifact from the ECG and determines whether a shockable rhythm is present in the filtered ECG. A recommendation of whether to administer a defibrillation shock to the patient is output by the monitor-defibrillator. In some cases, the monitor-defibrillator automatically charges in response to determining that the defibrillation shock is indicated. The user determines when and whether to administer the defibrillation shock based on the recommendation.

In some examples, the monitor-defibrillator incorrectly determines whether the patient is experiencing a shockable rhythm when the patient is moved, jostled, or otherwise experiences acceleration due to a cause other than chest compressions. These movements are common in various operational environments wherein a patient is transported, e.g., onto a cot, off of the cot, in a moving ambulance, or the like. These non-CPR movements are incompletely or incorrectly filtered by techniques designed to remove chest compression artifact, which leads to incorrect recommendations in some cases. According to various implementations described herein, the monitor-defibrillator automatically disables chest compression artifact filtering, or hides the recommendation, based on the detection of irregular movement of the patient. Thus, the monitor-defibrillator prevents the user from being misled by incorrect recommendations.

In some cases, the monitor-defibrillator calculates a certainty of a recommended shock decision. The certainty is based on detected movement (e.g., irregular movement), in some examples. According to some implementations, the certainty is based on filtered and/or unfiltered ECG itself. In some cases, the certainty is output with the recommendation, thereby providing the user with additional context for whether the defibrillation shock is recommended.

Some examples also relate to tracking the use of chest compression artifact filtering. In some examples, the monitor-defibrillator stores indications of whether chest compression artifact filtering is activated or deactivated, ECG data, indications of defibrillation shocks administered, and the like. Accordingly, an evaluator performing post-event analysis is able to determine whether the chest compression artifact filtering was or would have been accurate and/or whether the user would have administered a different treatment (e.g., a defibrillation shock or no defibrillation shock) if the chest compression artifact filtering was enabled or disabled.

Particular examples will now be described with reference to the accompanying figures. The scope of this disclosure includes individual examples described herein as well as any combination of the examples, unless otherwise specified.

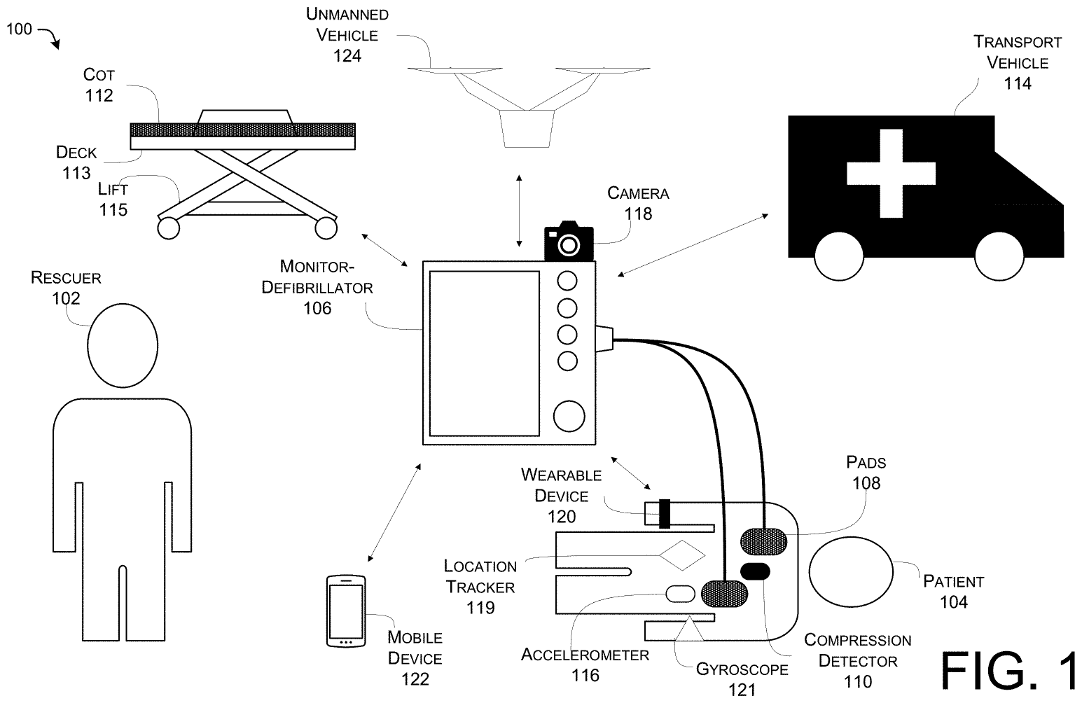

FIG. 1 illustrates an example of an emergency environment 100 in which a rescuer 102 is assisting a patient 104 in cardiac arrest. The rescuer 102 is a trained medical provider, such as an emergency responder, a public safety officer (e.g., a police officer, fireperson, or the like). The emergency environment 100, in various cases, is outside of a care facility, such as a clinical environment, a hospital, or any other type of environment wherein nonportable medical devices and highly trained care providers would be present.

Upon arriving at the emergency environment 100, the rescuer 102 uses a monitor-defibrillator 106 to monitor the patient 104. The rescuer 102 connects pads 108 to the chest of the patient 104. The pads 108 are in contact with the skin of the patient 104, according to various implementations. For instance, each of the pads 108 are attached to the skin of the patient 104 by an adhesive and/or a flexible substrate attached to the patient. Although only two pads 108 are illustrated in FIG. 1, some examples include more than two pads 108 connected to the patient 104. The pads 108 are connected to the monitor-defibrillator 106 by wired connections.

The monitor-defibrillator 106 detects an ECG of the patient 104 based on an electrical potential between at least two detection electrodes within the pads 108. In various examples, the monitor-defibrillator 106 displays or otherwise outputs the ECG of the patient 104 to the rescuer 102. The rescuer 102 evaluates the condition of the patient 104 based on the ECG, for example.

In various examples, the rescuer 102 administers chest compressions to the patient 104. According to some implementations, the rescuer 102 administers the chest compressions via a compression detector 110 that is placed on the chest of the patient 104. For instance, the rescuer 102 positions one or both hands on the compression detector 110 and presses on the compression detector 110 in order to administer chest compressions to the patient 104. The compression detector 110 includes a compression sensor (e.g., a force sensor configured to detect the chest compressions), one or more accelerometers, a gyroscope, or a combination thereof. The compression detector 110 detects the chest compressions administered to the patient 104. In various examples, the compression detector 110 provides a signal indicative of the chest compressions to the monitor-defibrillator 106. For example, the compression detector 110 is connected to the monitor-defibrillator 106 via a wireless and/or wired connection and transmits the signal to the monitor-defibrillator 106 over the wireless and/or wired connection. In some cases, the monitor-defibrillator 106 identifies a timing of the chest compressions based on the signal provided by the compression detector 110. In some examples, the monitor-defibrillator 106 determines a depth of the chest compressions administered to the patient 104 based on the signal provided by the compression detector 110. In some cases, the chest compressions are administered by a mechanical chest compression device (not illustrated). In some cases, the monitor-defibrillator 106 identifies timing of the chest compressions based on information provided by the mechanical chest compression device.

According to various implementations, the chest compressions generate noise in the ECG. The noise is at least partly based on jostling or movement of the pads 108 on the skin of the patient 104, for example. An artifact is present in the detected ECG based on the chest compressions. The artifact makes the ECG output by the monitor-defibrillator 106 difficult for the rescuer 102 to evaluate, in some cases. For instance, the rescuer 102 has difficulty determining whether a shockable rhythm is present in the ECG output by the monitor-defibrillator 106.

In some examples, the rescuer 102 activates an advisory mode (also referred to as an “analysis mode”) in which the monitor-defibrillator 106 analyzes the ECG and provides a recommendation indicating whether to administer a defibrillation shock to the patient 104 based on the analysis. For instance, the monitor-defibrillator 106 receives, from the rescuer 102, an input signal selecting the advisory mode. The input signal is received by an input device of the monitor-defibrillator 106. For example, the rescuer 102 presses a button of the monitor-defibrillator 106 or touches a touchscreen of the monitor-defibrillator 106. The monitor-defibrillator 106 activates the advisory mode based on the input signal, in some cases.

When the advisory mode is active, the monitor-defibrillator 106 selects and analyzes a segment of the ECG. The monitor-defibrillator 106 generates a filtered ECG by removing the chest compression artifact from the ECG of the patient 104. In some cases, the monitor-defibrillator 106 applies a digital filter that selectively removes or reduces one or more frequency bands corresponding to the chest compressions administered to the patient 104. The frequency bands, in some cases, include the chest compression frequency as well as one or more harmonics of the chest compression frequency.

The monitor-defibrillator 106 determines the frequency and/or timing of the chest compressions administered to the patient 104 in any of a variety of ways. In some cases, the monitor-defibrillator 106 determines when the chest compressions are administered based on the signal from the compression detector 110. In some examples, the monitor-defibrillator 106 detects an electrical impedance between two or more of the detection electrodes within the pads 108 and determines when the chest compressions are administered based on the electrical impedance.

According to various implementations, the monitor-defibrillator 106 uses the filtered ECG to determine whether the patient 106 is experiencing a shockable heart rhythm, such as VF or pulseless V-Tach. In some examples, the monitor-defibrillator 106 refrains from outputting the filtered ECG to the rescuer 102. For instance, even when the monitor-defibrillator 106 is capable of identifying the presence of the shockable heart rhythm in the filtered ECG, the filtered ECG may look unfamiliar to the rescuer 102. Thus, the monitor-defibrillator 106, in some examples, refrains from outputting the filtered ECG in order to avoid confusion by the rescuer 102.

In some examples, the monitor-defibrillator 106 generates a shock index based on the filtered ECG. The shock index, for example, corresponds to a likelihood that the filtered ECG includes the shockable rhythm. In some cases, a positive shock index indicates that the filtered ECG is more likely than not to include a shockable rhythm and a negative shock index indicates that the filtered ECG is more likely than not to include a non-shockable rhythm. In some cases, the monitor-defibrillator 106 generates the shock index based on one or more parameters of the filtered ECG. For instance, the shock index is based on an amplitude-spectral area (AMSA) value of the filtered ECG.

In various implementations, the monitor-defibrillator 106 concludes whether the patient 104 is experiencing a shockable rhythm by comparing the shock index to one or more thresholds. For instance, the monitor-defibrillator 106 compares the shock index to an upper threshold and a lower threshold. If the shock index exceeds both the upper threshold and the lower threshold, the monitor-defibrillator 106 determines that a shockable rhythm is present in the ECG and comes to a “shockable decision.” If the shock index is lower than both the upper threshold and the lower threshold, the monitor-defibrillator 106 determines that a shockable rhythm is not present in the ECG and comes to a “nonshockable decision.” If the shock index is between the upper threshold and the lower threshold, the monitor-defibrillator 106 comes to an “indeterminate decision.” The indeterminate decision means that the monitor-defibrillator 106 is unable to conclude whether the shockable rhythm is present with a sufficient level of certainty. The level of certainty, in some cases, is predetermined and/or selected by the rescuer 102.

In the advisory mode, the monitor-defibrillator 106 outputs the shock decision to the rescuer 102. In some cases, the monitor-defibrillator 106 performs filtering and/or determines the shock decision even when the advisory mode is inactive. However, the monitor-defibrillator 106 refrains from outputting the shock decision to the rescuer 102 until the advisory mode is activated. In some cases, the monitor-defibrillator 106 automatically begins charging a capacitor upon determining that the shockable rhythm is present, and in some examples automatically begins charging the capacitor when the analysis result is indeterminate.

According to some examples, the rescuer 102 applies a defibrillation shock to the patient 102 based on the recommendation. For example, the pads 108 include defibrillation electrodes. In response to receiving an input signal from the rescuer 102, the monitor-defibrillator 106 charges a capacitor and/or discharges the capacitor across the defibrillation electrodes, thereby applying a defibrillation shock to the heart of the patient 104. In some cases in which the patient 104 is experiencing a shockable arrhythmia, the defibrillation shock may end the shockable arrhythmia and address the source of the cardiac arrest. In some cases, the heart of the patient 104 begins to function effectively, thereby resulting in a return of spontaneous circulation (ROSC).

Although the chest compression artifact filtering techniques described above are powerful tools for accurately determining whether the patient 104 is exhibiting a shockable rhythm during CPR, these techniques ineffectively filter artifact from irregular movement and, consequently, an erroneous shock decision can result when the patient 104 is experiencing irregular movement. In some cases, the erroneous shock decision causes the rescuer 102 to administer a defibrillation shock when the patient 104 is not exhibiting a shockable rhythm, which can physically harm (e.g., burn) the patient 104, exacerbate a non-shockable arrhythmia experienced by the patient 104, or even cause the patient 104 to experience an arrhythmia. In some examples, the erroneous shock decision causes the rescuer 102 to refrain from administering a defibrillation shock when the patient is exhibiting a shockable rhythm, which can delay or fully prevent treatment of the patient's 104 cardiac arrest. Thus, erroneous shock decisions can have serious consequences on the care of the patient 104.

As used herein, the terms “irregular movement,” “non-CPR movement,” “irregular motion,” and their equivalents, can refer to a speed, a velocity, an acceleration, a jerk, or any other type of movement, that is misaligned, independent, or otherwise noncorrelated with chest compressions. In various examples, a frequency spectrum representation of an irregular movement is distributed over frequencies that are outside of a fundamental frequency or harmonics of chest compressions.

Irregular movements are common in emergency scenarios. For example, the patient 104 experiences irregular movement when physically transferred onto and/or off of a cot 112. The cot 112 includes a deck 113 and a lift 115. In some examples, deck 113 includes a cushion on which the patient 104 is supported. The lift 115 is configured to raise, lower, or otherwise vertically support the deck 113. The patient 104 experiences irregular movement when the cot 112 is moved (e.g., rolled, carried, or the like) while the cot 112 is supporting the patient 104.

A transport vehicle 114 is a source of irregular movement, in some cases. The patient 104 experiences irregular movement when physically transferred onto or off of the transport vehicle 114. The patient 104 also experiences irregular movement when the transport vehicle 114 is moving while the patient 104 is transported by the transport vehicle 114.

To avoid misleading the rescuer 102, the monitor-defibrillator 106 selectively outputs the recommendation (e.g., only) when the patient 104 is experiencing less than a threshold amount of irregular movement. In contrast, the monitor-defibrillator 106 refrains from outputting the recommendation when the patient 104 is experiencing greater than or equal to the threshold amount of irregular movement. In some examples, the monitor-defibrillator 106 refrains from filtering and/or generating the recommendation when a certain amount (e.g., greater than or equal to the threshold amount) of irregular movement is detected, even when the advisory mode is activated. In some examples, the monitor-defibrillator provides a visual, audio, or haptic feedback output to the rescuer 102 to indicate that the recommendation is being withheld and the reason the recommendation is being withheld (e.g., that the irregular movement has been detected).

In some examples, the monitor-defibrillator 106 detects the irregular movement of the patient 104 based on a transthoracic impedance of the patient 104. For example, the monitor-defibrillator 106 detects an impedance of the patient 104 based on an impedance between at least two detection electrodes within the pads 108. In various examples, the monitor-defibrillator 106 detects the irregular movement based on a change in the impedance over time. In some cases, the monitor-defibrillator 106 detects the irregular movement based on the impedance when the patient 104 is not receiving chest compressions. In particular examples, the monitor-defibrillator 106 detects the chest compressions based on the impedance of the patient 104.

The monitor-defibrillator 106 detects the irregular movement of the patient 104 based on one or more motion sensors in the emergency environment 100. The motion sensor(s) include, for instance, an accelerometer 116. The accelerometer 116 detects its own acceleration in space. In some cases, the accelerometer 116 is attached to the patient 104, such that by detecting its own acceleration, the accelerometer 116 detects an acceleration of the patient 104. The accelerometer 116 is attached, for example, to a limb of the patient 104 (e.g., an arm or a leg), the chest of the patient 104, the head of the patient 104, the abdomen of the patient 104, a hand of the patient 104, a foot of the patient 104, some other part of the patient 104, or any combination thereof. In some examples, the accelerometer 116 is attached to the skin of the patient 104 by an adhesive, by being attached to a substrate (e.g., a flexible substrate) that is adhered to the skin of the patient 104, or the like. For instance, the accelerometer 116 is part of a patch device that is adhered to the patient 104. In some implementations, the accelerometer 116 is attached to a band (e.g., a wrist-band, an arm-band, a leg-band, a chest-band, etc.) that is disposed around a portion of the patient 104. In various cases, the accelerometer 116 is connected to the monitor-defibrillator 106 by a wired and/or wireless connection. The accelerometer 116 provides, to the monitor-defibrillator 106, a signal indicative of the acceleration of the patient 104 over the wired and/or wireless connection.

In some cases, the motion sensor(s) includes a camera 118. The camera 118 captures one or more images of the patient 104. For example, the camera 118 captures a video of the patient 104. The camera 118 includes, for instance, an array of electromagnetic sensors that detect electromagnetic waves reflected by the patient 104. In some cases, the camera 118 includes a source that emits at least a portion of the electromagnetic waves reflected by the patient 104. The camera 118 generates data indicative of the image(s) based on the detected light. In various examples, the monitor-defibrillator 106 detects movement of the patient 104 based on the image(s) captured by the camera 118. In some implementations, the monitor-defibrillator 106 detects the movement of the patient 104 by detecting a difference in positions of any portion of the patient 104 in consecutive frames of the video, by detecting blur in the image(s), or the like. According to some examples, the camera 118 is attached to the patient 104 and captures one or more images of the emergency environment 100. The monitor-defibrillator 106 detects movement of the patient 104 by detecting a difference between images of the emergency environment 100, blur in the image(s) of the emergency environment 100, or the like. In various cases, the camera 118 is connected to the monitor-defibrillator 106 by a wired and/or wireless connection. The camera 118 provides, to the monitor-defibrillator 106, a signal indicative of the image(s) and/or video of the patient 104 or the emergency environment 100 over the wired and/or wireless connection.

In particular implementations, the motion sensor(s) includes a location tracker 119. The location tracker 119 includes a digital and/or analog circuit that detects a location of the location tracker 119 relative to the earth's surface. When the position of the location tracker 119 is associated with a location of the patient 104, the location tracker 119 detects the location of the patient 104. In some examples, the location tracker 119 receives signals transmitted by satellites and detects the location based on the received signals. In various examples, the satellites are part of the Global Positioning System (GPS), the Global Navigation Satellite System (GLONASS), BeiDou Navigation Satellite System, the Galileo positioning system, NavIC, Quasi-Zenith Satellite System (QZSS), or any combination thereof. In some cases, the location tracker 119 detects a movement of the patient 104 based on a change in the location of the location tracker 119 and/or the patient 104. The location tracker 119 is attached to the patient 104, in some examples. For instance, the location tracker 119 is adhered to the patient 104, attached to a substrate that is adhered to the patient 104, attached to a band that is wrapped around a portion of the patient 104, or any combination thereof. In various cases, the location tracker 119 is connected to the monitor-defibrillator 106 by a wired and/or wireless connection. In some cases, the monitor-defibrillator 106 includes the location tracker 119. The location tracker 119 provides, to the monitor-defibrillator 106, a signal indicative of the location and/or movement of the patient 104 over the wired and/or wireless connection.

In some examples, the motion sensor(s) includes a gyroscope 121. The gyroscope 121 detects a change in orientation and/or angular velocity of the patient 104. In some cases, the gyroscope 121 includes a gimbal that is attached to the patient 104 and a second gimbal and/or some reference object (e.g., the cot 112 or the emergency vehicle 114). The gyroscope 121 detects any change with respect to the relative orientation of the patient 104 and/or reference object. In various cases, the gyroscope 121 is connected to the monitor-defibrillator 106 by a wired and/or wireless connection. The gyroscope 121 provides, to the monitor-defibrillator 106, a signal indicative of the angular movement of the patient 104 over the wired and/or wireless connection.

In various implementations, the motion sensor(s) are integrated into one or more devices or objects in the emergency environment 100. For instance, the monitor-defibrillator 106 includes the accelerometer 116, the camera 118, the location tracker 119, the gyroscope 121, or any combination thereof. In some examples, the motion sensor(s) detect motion of the monitor-defibrillator 106. The motion of the monitor-defibrillator 106, in various implementations, is assumed to correspond to the motion of the patient 104.

In some examples, the cot 112 includes the accelerometer 116, the camera 118, the location tracker 119, the gyroscope 121, or any combination thereof. According to some implementations, the cot 112 includes multiple accelerometers 116, multiple cameras 118, multiple location trackers 119, or any combination thereof. For instance, one or more accelerometers 116 are integrated with or attached to a support rail of the cot 112, one or more wheels of the cot 112, one or more legs of the cot 112, the lift 115 of the cot 112, the deck 113 of the cot 112, a restraint of the cot 112, some other element of the cot 112, or any combination thereof. Any movement of the cot 112 detected by the accelerometer(s) 116 are assumed to correspond to movement of the patient 104 when the patient 104 is supported by the cot 112. In some cases, the camera 118 is attached to or integrated with the support rail of the cot 112, the headrest of the cot 112, some other element of the cot 112, or any combination thereof.

In some examples, the cot 112 includes one or more gyroscopes 121. For instance, a first gimbal of the gyroscope 121 is attached to the deck 113 and a second gimbal of the gyroscope 121 is attached to the lift 115. In some cases, multiple (e.g., three or more) gyroscopes 121 are similarly attached between the deck 113 and the lift 115. In some cases, the gyroscope(s) 121 dampen the effect of movement of the lift 115 on the deck 113, thereby reducing the movement of the patient 104 when the patient 104 is supported by the deck 113. The gyroscope(s) 121 is also configured to detect a change in orientation between the deck 113 and the lift 115. The change in orientation between the deck 113 and the lift 115 corresponds to movement of the patient 104 when the patient 104 is supported by the cot 112, in various examples.

According to some examples, the transport vehicle 114 includes one or more of the accelerometers 116, one or more of the cameras 118, one or more of the location trackers 119, one or more of the gyroscopes 121, or any combination thereof. According to some examples, the motion sensor(s) includes a speedometer of the transport vehicle 114. For instance, the monitor-defibrillator 106 connects to a computing system in the transport vehicle 114 that transmits, to the monitor-defibrillator 106, a signal indicative of the speed that the transport vehicle 114 is traveling. In some cases, the movement of the transport vehicle 114 is presumed to correspond to movement of the patient 104 when the patient is located inside of the transport vehicle 114.

In some examples, the patient 104 is wearing a wearable device 120 that includes the accelerometer(s) 116, the camera(s) 118, the location tracker(s) 119, the gyroscope(s) 121, or any combination thereof. The wearable device 120 is attached to the patient 104 and/or the rescuer 102. For instance, the wearable device 120 is a smartwatch attached to a wristband that is worn around a wrist of the patient 104. In some cases, the camera(s) 118 in the smartwatch capture image(s) of the emergency environment 100, which are used by the monitor-defibrillator 106 to determine motion of the patient 104.

The rescuer 102 and/or the patient 104 has a mobile device 122, which includes the accelerometer(s) 116, the camera(s) 118, the location tracker(s) 119, the gyroscope(s) 121, or any combination thereof, in some implementations. The mobile device 122 is worn, held, or otherwise attached to the rescuer 102 and/or the patient 104. In some examples, the mobile device 122 is a cell phone. In examples in which the rescuer 102 and the patient 104 are touching or located in the same sub-environment (e.g., both the rescuer 102 and the patient 104 are located inside of the transport vehicle 114), the motion of the rescuer 102 is presumed to correspond to the motion of the patient 104.

As illustrated, the emergency environment 100 further includes an unmanned vehicle 124 that, in some implementations, includes the camera(s) 118 and/or location tracker(s) 119. In some cases, the unmanned vehicle 124 is an unmanned aerial vehicle (UAV). According to particular implementations, the unmanned vehicle 124 is configured to follow the rescuer 102 and/or the patient 104 around the emergency environment 100. The unmanned vehicle 124 captures image(s) of the patient 104 via the camera(s) 118 and transmits the image(s) to the monitor-defibrillator 106, for instance.

In some cases, the accelerometer 116 and/or the gyroscope 121 is integrated into the compression detector 110. For example, the accelerometer 116 provides a signal indicative of the acceleration of the compression detector 110 to the monitor-defibrillator 106, which enables the monitor-defibrillator 106 to both detect chest compressions administered to the patient 104 and detect irregular motion of the patient 104.

In various implementations, the monitor-defibrillator 106 utilizes multiple motion sensors to identify motion at different points along the body of the patient 104. For example, a first accelerometer 116 is attached to the right leg of the patient 104, a second accelerometer 116 is attached to the left leg of the patient 104, a third accelerometer 116 is attached to the head of the patient 104, a fourth accelerometer 116 is attached to the chest of the patient 104, a fifth accelerometer 116 is attached to the head of the patient, or any combination thereof. In some instances, a first camera 118 captures image(s) of a first portion of the patient 104 (e.g., the head of the patient 104), a second camera captures image(s) of a second portion of the patient 104 (e.g., the chest of the patient 104), or a combination thereof.

The monitor-defibrillator 106 selectively generates and/or outputs the shock recommendation based on the motion of the patient 104. In various examples, the motion of the patient 104 corresponds to a velocity, an acceleration, a jerk, or any combination thereof, of any portion of the patient 104. The monitor-defibrillator 106 determines the motion of the patient 104 based on motion detected by the motion sensor(s). For example, the monitor-defibrillator 106 determines at least one position of the patient 104 over time and determines the motion based on a first-, second-, or higher-order differential of the position.

In some cases, the monitor-defibrillator 106 specifically identifies irregular motion of the patient 104. Chest compressions cause some movement of the patient 104 but are adequately filtered from the ECG by various filtering mechanisms described herein. Chest compressions cause regular motion, which is motion corresponding to one or more frequency peaks. In contrast, irregular motion is distributed widely across the frequency spectrum. In some cases, the monitor-defibrillator 106 identifies irregular motion by applying a high-pass filter (e.g., with a cutoff frequency of 3 Hz) to data indicative of the motion of the patient 104. In some examples, the monitor-defibrillator 106 presumes that any motion of the patient 104 detected between consecutive chest compressions applied to the patient 104 is irregular motion. For instance, the monitor-defibrillator 106 identifies time periods during which chest compressions are not applied to the patient 104, such as time periods between chest compressions. Motion detected during those time periods is assumed to include irregular motion, in some cases.

In some examples, the chest compression motion as measured by the compression detector 110 is compared to the various motion sensors (accelerometers, gyroscopes, cameras, etc.) and significant deviation between the two or more sensors is utilized to determine the presence of non-chest compression motion. For example, if the compression detector 110 detects chest compression at a particular time or frequency, and one or more of the other motion sensors detects motion at a frequency that is different than the particular time or frequency, the monitor-defibrillator 106 is configured to detect the irregular motion.

In various examples, the monitor-defibrillator 106 compares the irregular motion of the patient 104 to a threshold. If the irregular motion is less than the threshold, the monitor-defibrillator 106 will generate and/or output the shock recommendation based on the ECG. On the other hand, if the irregular motion is greater than or equal to the threshold, the monitor-defibrillator 106 will refrain from generating and/or outputting the shock recommendation based on the ECG. Accordingly, the monitor-defibrillator 106 is prevented from outputting a misleading shock recommendation to the rescuer 102.

In particular implementations, the monitor-defibrillator 106 outputs the shock recommendation in response to a manual override. For example, in the advisory mode, the monitor-defibrillator 106 outputs a warning indicating that significant irregular motion has been detected and/or the advisory mode is unable to determine an accurate shock recommendation. In some circumstances, the rescuer 102 may nevertheless seek the benefit of the shock recommendation. For example, the rescuer 102 provides an input signal that is received by the monitor-defibrillator 106. In response to receiving the input signal, the monitor-defibrillator outputs the shock recommendation despite the detected irregular motion.

In some implementations, the monitor-defibrillator 106 calculates and/or outputs an accuracy of the shock decision based on the detected irregular motion of the patient 104. For example, if the irregular motion is greater than or equal to the threshold, the monitor-defibrillator 106 will output the recommendation with an indication of the accuracy of the recommendation. In some cases, the monitor-defibrillator 106 outputs the indication of the accuracy regardless of whether the irregular motion is above the threshold. In some cases, the monitor-defibrillator 106 determines the accuracy based on the ECG (e.g., based on the shock index) and/or the irregular motion detected by the motion sensor(s). For example, the indication of the accuracy of the recommendation is expected to decrease as the amount of irregular motion is increased. Thus, the monitor-defibrillator 106 provides the rescuer 102 with additional information that enables the rescuer 102 to make an informed decision about whether to follow the recommendation, even if the recommendation is likely to be inaccurate.

As discussed above, the monitor-defibrillator 106 selectively outputs a recommendation based on ECG filtering for a variety of reasons. In some cases, the rescuer 102 manually activates or deactivates the advisory mode. In some examples, the monitor-defibrillator 106 automatically hides and/or refrains from generating a recommendation based on detected irregular motion of the patient 104. In some examples, the monitor-defibrillator 106 stores data indicating when and whether shock recommendations are generated and output. In some cases, the monitor-defibrillator 106 stores data indicating whether chest compression filtering is activated or deactivated. The monitor-defibrillator 106 also stores data indicative of the ECG, impedance, administered defibrillation shocks, or a combination thereof. In some cases, the monitor-defibrillator 106 stores timestamps associated with different events, such as data indicative of when the chest compression filtering is active, when the defibrillation shocks are administered, and so on. In some cases, the stored data is accessed by a user for post-event analysis. Accordingly, the user is able to conclude whether an erroneous recommendation was output to the rescuer 102 and caused the rescuer 102 to administer a contraindicated treatment to the patient 104, whether a correct recommendation was hidden and the rescuer 102 administered a contraindicated treatment to the patient 104, or the like.

In various implementations, the monitor-defibrillator 106 is configured to warn the rescuer 102 about a contraindicated treatment when the monitor-defibrillator 106 is operating in “manual mode.” For example, the rescuer 102 may view the ECG of the patient 104 output on the monitor-defibrillator 106 and perform a manual rhythm analysis on the ECG. In some examples in which the rescuer 102 determines that the ECG of the patient 104 exhibits a shockable rhythm (e.g., VF or pulseless V-Tach), the rescuer 102 operates the monitor-defibrillator 106 to administer an electrical shock to the patient 104. However, if the rescuer 102 determines that the ECG of the patient 104 does not exhibit the shockable rhythm, the rescuer 102 refrains from operating the monitor-defibrillator 106 to administer the electrical shock to the patient 104. Irregular motion may reduce the accuracy of a manual analysis of the ECG by the rescuer 102. In some cases, the monitor-defibrillator 106 outputs a message indicating that ECG analysis is unreliable upon detecting irregular motion. In various examples, the monitor-defibrillator 106 outputs a message indicating that any automated analysis performed by the monitor-defibrillator 106 conflicts with a manual action (e.g., to administer the electrical shock) performed by the rescuer 102.

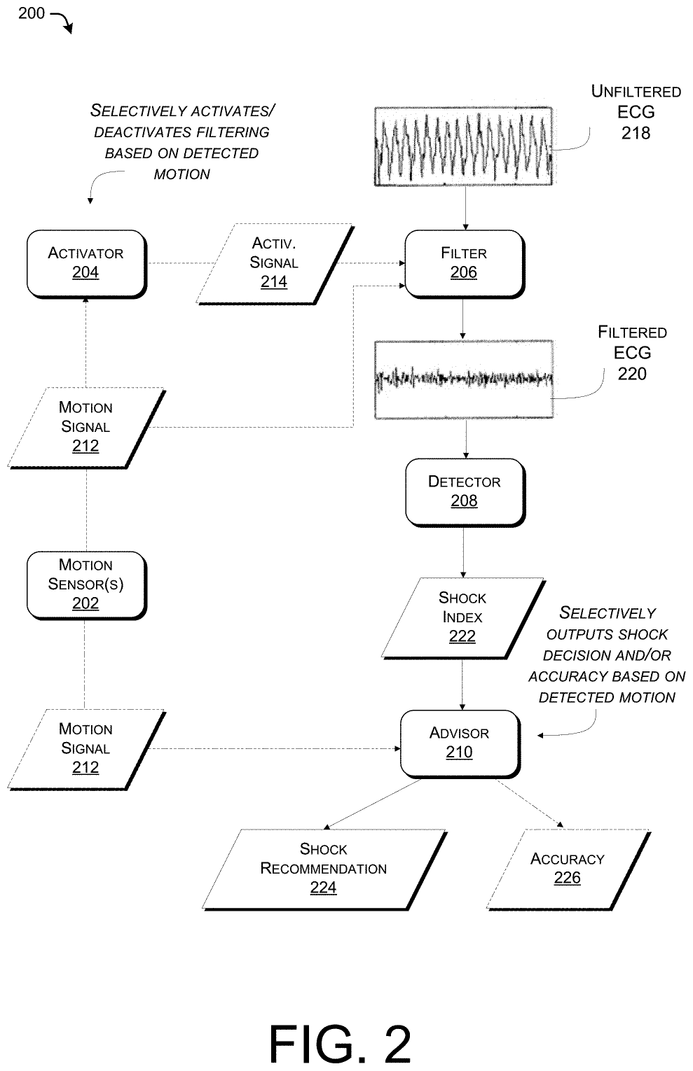

FIG. 2 illustrates example signaling 200 associated with accommodating for irregular motion of a patient as a monitor-defibrillator is operating in manual mode. The signaling 200 is between at least one motion sensor 202, an activator 204, a filter 206, a detector 208, and an advisor 210. In various examples, the motion sensor(s) 202 include the accelerometer 116, the camera 118, the location tracker 119, or any combination thereof, described above with reference to FIG. 1. According to some implementations, the activator 204, the filter 206, the detector 208, and the advisor 210 are software and/or hardware elements that are implemented by and/or integrated with the monitor-defibrillator 106 described above with reference to FIG. 1.

The motion sensor(s) 202 detects a motion of an individual (e.g., the patient 104 described above with reference to FIG. 1) and generates a motion signal 212 based on the detected motion. The motion sensor(s) 202 transmits the motion signal to the activator 204.

The activator 204 determines an irregular motion of the patient 104 based on the motion signal 212. For example, the activator 204 can filter out at least a portion of the motion signal 212 that corresponds to motion associated with chest compressions administered to the patient 104. In some cases, the activator 204 applies a high-pass filter, an FIR filter, a Kalman filter, a comb filter, or a combination thereof, to the motion signal 212. In some implementations, the activator 204 identifies one or more time windows that correspond to chest compressions administered to the individual and selects a portion of the motion signal 212 between the identified time windows. The activator 204 identifies the irregular motion of the individual based on the resultant filtered and/or portions of the motion signal 212. In some examples, the irregular motion is a velocity, an acceleration, a jerk, or some other higher order movement, of the individual.

In some examples, the activator 204 compares the irregular motion of the patient 212 to a threshold. The activator 204 generates an activation signal 214 based on the comparison. For example, the activator 204 selectively generates the activation signal 214 when the irregular motion is less than a threshold. The activator 204 transmits the activation signal 214 to the filter 206.

The filter 206 receives an unfiltered ECG 218 of the individual. When the filter 206 receives the activation signal 214, the filter 206 generates a filtered ECG 220 by removing a chest compression artifact from the unfiltered ECG 218. In some examples, the filter 206 removes the filtered ECG 220 by applying one or more filters to the unfiltered ECG 218, such as a high-pass filter (e.g., with a cutoff frequency of 3 Hz, such that a fundamental chest compression frequency is rejected), a Kalman filter, a comb filter (e.g., with one band including a frequency of the chest compressions administered to the individual and other bands including harmonics of the frequency).

According to some instances, the motion sensor 202 further provides the motion signal 212 to the filter 206. The filter 206 removes, from the unfiltered ECG 218, additional motion artifact (e.g., an irregular motion artifact) based on the motion signal 212, in various examples.

In various implementations, the filter 206 provides the filtered ECG 220 to the detector 208. In some cases in which the filter 206 is part of or connected to a monitor-defibrillator, the monitor-defibrillator displays the unfiltered ECG 218 rather than the filtered ECG 220. This is because although a rhythm depicted in the filtered ECG 220 is discernible to a computing system, the filtered ECG 220 looks unlike a natural ECG, and thus could be confusing to a rescuer operating the monitor-defibrillator.

The detector 208 determines whether a shockable rhythm is present in the filtered ECG 220. The detector 208 generates a shock index 222 based on the filtered ECG 220. The shock index 222, for instance, is indicative of a likelihood that the filtered ECG 220 includes a shockable rhythm. The detector 208 provides the shock index 222 to the advisor 210.

The advisor 210 generates a shock recommendation 224 based on the shock index 222. In some examples, the advisor 210 compares the shock index 222 to an upper threshold and a lower threshold. If the shock index 222 is greater than both the upper threshold and the lower threshold, then the advisor 210 generates the shock recommendation 224 to indicate that a defibrillation shock should be administered to the individual. If the shock index 222 is less than both the upper threshold and the lower threshold, then the advisor 210 generates the shock recommendation 224 to recommend that a defibrillation shock should not be administered to the individual. If the shock index 222 is between the upper threshold and the lower threshold, then the advisor generates the shock recommendation 224 to indicate an indeterminate shock decision.

In some examples, the advisor 210 further generates and outputs an indication of the accuracy 226 of the shock recommendation 224. In examples in which the shock index 222 corresponds to the percentage certainty that the shockable rhythm is present in the filtered ECG 220, the advisor 210 generates the accuracy 226 based on the shock index 222. According to various examples, the advisor 210 receives the motion signal 212 from the motion sensor(s) 202. The advisor 210 further generates the accuracy 226 based on the motion signal 212, in some cases. For instance, the advisor 210 decreases the accuracy 226 based on the type and/or magnitude of motion sensed by the motion sensor(s) 202.

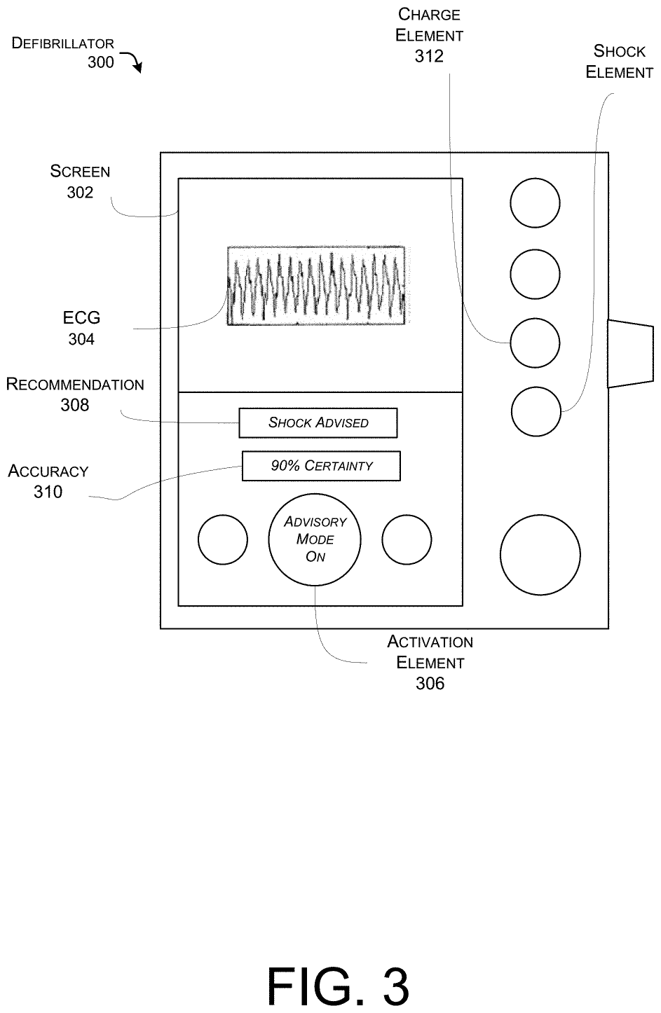

FIG. 3 illustrates an example of a defibrillator 300 visually displaying ECG-related data. The defibrillator 300 is, for example, the monitor-defibrillator 106 described above with reference to FIG. 1. In the example of FIG. 3, the defibrillator 300 is operating in manual mode and advisory mode. The defibrillator 300 displays the ECG-related data on a screen 302. In some examples, the screen 302 is a touchscreen.

The screen 302 displays an ECG 304 of an individual being monitored. The ECG 304 is unfiltered, in various cases. For example, the ECG 304 displayed on the screen 302 is the unfiltered ECG 220 described above with reference to FIG. 2. In some cases, the ECG 304 is displayed on an upper portion of the screen 302. The ECG 304 is obtained as chest compressions are administered to the individual, such that a chest compression artifact is present in the ECG 304.

Although not illustrated in FIG. 3, in some cases, the defibrillator 300 is configured to output the ECG 304 with multiple waveforms corresponding to various leads. For instance, the ECG 306 includes twelve waveforms, arranged in rows and/or columns, corresponding to a 12-lead signal. In various examples, the 12-lead ECG 304 is obtained from the patient during a time interval when the patient is not receiving chest compressions. The 12-lead signal, for example, assists a user with diagnosing a condition of the patient, such as ST-Elevation Myocardial Infarction (STEMI). In some cases, the defibrillator 300 is configured to refrain from outputting the 12-lead ECG 304 in response to detecting irregular motion.

In the example of FIG. 3, the screen 302 also displays an activation element 306. The activation element 306 indicates whether the advisory mode is active or inactive. In some examples, the activation element 306 is selectable, such that a user can activate and/or deactivate the advisory mode by entering a user input signal associated with the activation element 306 into the defibrillator 300. For instance, the screen 302 is a touch screen and the defibrillator 300 activates and/or deactivates the advisory mode based on a touch signal received by one or more touch sensors corresponding to the area of the activation element 306 displayed on the screen 302.

According to various implementations of the present disclosure, the defibrillator 300 selectively activates the advisory mode based on irregular motion of the individual. For example, even if the activation element 306 is selected based on the user input signal, the defibrillator 300 nevertheless deactivates the advisory mode if the irregular motion of the individual is greater than or equal to a threshold. In some instances, the defibrillator 300 activates the advisory mode when the activation element 306 is selected and the irregular motion of the individual is less than the threshold.

When the advisory mode is active, the defibrillator 300 analyzes the ECG 304 and determines whether the ECG 304 exhibits a discernable shockable rhythm. In some cases, the defibrillator 300 selects a segment of the ECG 304 and filters the chest compression artifact from the selected segment using one or more filtering techniques. In some examples, the defibrillator 300 generates a shock index based on the filtered ECG 304 and determines whether the shockable rhythm is present by comparing the shock index to one or more thresholds. The defibrillator 300 outputs a recommendation 308 on the screen 302 based on the analysis of the ECG 304. In the example illustrated in FIG. 3, the defibrillator 300 determines that the ECG 304 exhibits a shockable rhythm (e.g., VF) and the recommendation 308 indicates that a shock is advised to treat the shockable rhythm of the individual.

In this example, the defibrillator 300 also outputs an indication of the accuracy 310 on the screen 302. The accuracy 310 indicates the certainty of the recommendation 308. In some cases, the defibrillator 300 selectively outputs the accuracy 310 in response to determining that the irregular motion of the individual is above a particular threshold. The defibrillator 300 generates the accuracy 310 based on the shock index and/or detected irregular motion of the individual. In some cases, the accuracy 310 is represented as a gauge indicating the certainty, a color indicating the certainty (e.g., green for greater than 70% certainty, red for less than 70% certainty, etc.), or any other graphical user interface element that shows a readily discernible certainty to the user of the defibrillator 300.

The defibrillator 300, in some cases, charges one or more capacitors in response to a user input signal and/or the determination that the shock is advised. For example, the defibrillator 300 charges the capacitor(s) in response to a charge element 312 receiving a user input signal. The charge element 312 is, for instance, a button. In some examples, the charge element 312 is a user-selectable graphical user interface element displayed on the screen 302. According to some implementations, the defibrillator 300 automatically charges the capacitor(s) in response to determining that the shockable rhythm is present in the ECG 304. In other implementations the defibrillator 300 automatically charges the capacitor when the analysis result is indeterminate.

Because the defibrillator 300 is operating in manual mode, the defibrillator 300 administers a defibrillation shock to the individual in response to an input signal from the user. For example, the defibrillator 300 outputs the defibrillation shock based on a user input signal received by a shock element 314. The defibrillator 300 outputs the defibrillation shock by discharging the charged capacitor(s). The shock element 314 is, for instance, a button. In some cases, the shock element 314 outputs a signal (e.g., a light signal) when the capacitor(s) is charged. In some implementations, the shock element 314 is a user-selectable graphical user interface displayed on the screen 302.

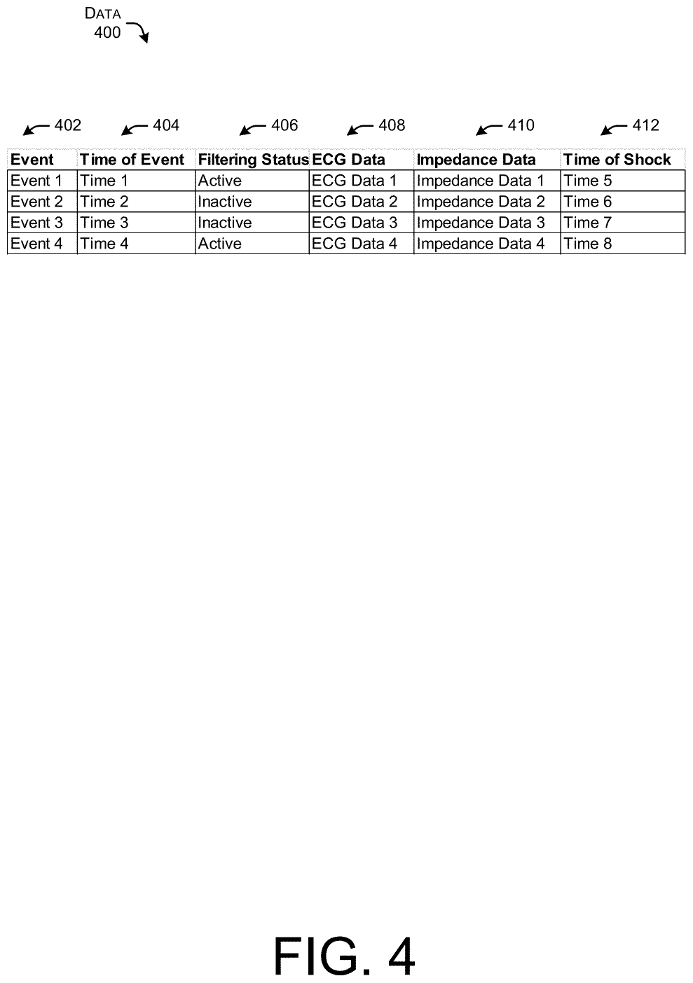

FIG. 4 illustrates an example of data 400 stored at least temporarily in memory of a defibrillator. For instance, the data 400 is stored in memory of the monitor-defibrillator 106 described above with reference to FIG. 1 and/or memory of the defibrillator 300 described above with reference to FIG. 3. The data 400 includes a table stored in a database, for example.

The data 400 includes multiple data fields, which are represented as columns in the table. In this example, the data fields include an event field 402, a time of event field 404, a filtering status field 406, an ECG data field 408, an impedance data field 410, and a time of shock field 412. The data 400 represents multiple events, which are arranged as rows in the table. An event, for instance, corresponds to a unique patient being treated by the defibrillator. The event field 402 includes a unique identifier (e.g., a number, a string, or the like) of each event. The time of event field 404 includes a time at which each event occurs, such as a time of the beginning of each event and/or a time of the end of each event. The filtering status field 406 includes a flag that indicates whether the advisory mode is active during each event. The ECG data field 408 includes ECG data that is obtained during each event. The impedance data field 410 includes impedance data that is obtained during each event. The time of shock field 412 includes times and/or flags indicating defibrillation shocks, if any, administered during each event.

In some implementations, the data 400 is used for post event analysis of the events. For example, a reviewer with access to the data 400 independently evaluates the ECG data for each event in the ECG data field 408 and/or the impedance data for each event in the impedance data field 410 in order to independently determine whether a defibrillation shock was indicated. By evaluating the time of shock field 412 for each event, the reviewer is able to determine whether a shock was correctly administered. For example, the reviewer determines whether a rescuer administered a defibrillation shock in events where the defibrillation was not indicated, determines whether a rescuer failed to administer a defibrillation shock in events where the defibrillation shock was indicated, or the like. The reviewer is also able to determine, based on the filtering status field 406, whether the rescuer would have correctly administered a defibrillation shock if the advisory mode was active. Thus, the reviewer is able to determine how well the advisory mode would improve patient care.

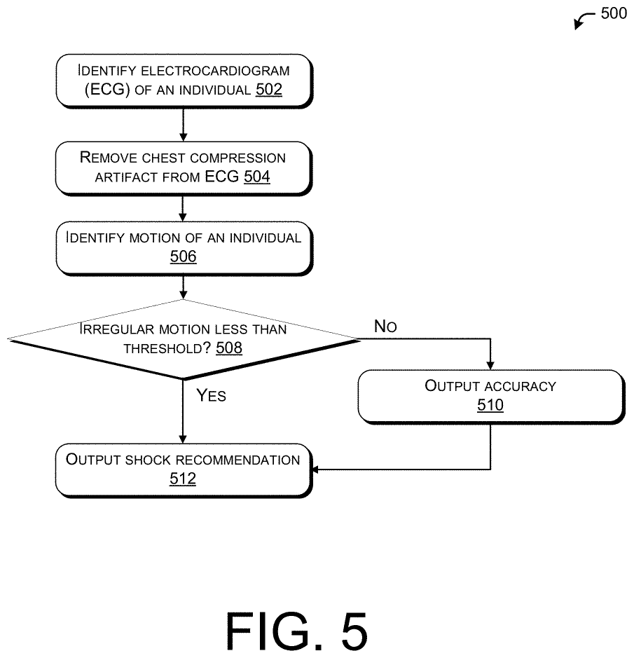

FIGS. 5 to 8 illustrate processes that can be performed according to various implementations described herein. Although the processes illustrated in FIGS. 5 to 8 are shown in particular orders, the processes can be performed in alternate orders according to some implementations.

FIG. 5 illustrates an example process 500 for selectively outputting an accuracy of a shock recommendation based on irregular motion detection. In various cases, the process 500 is performed by a medical device, such as the monitor-defibrillator 106 described above with reference to FIG. 1 and/or the defibrillator 300 described above with reference to FIG. 3.

At 502, the medical device identifies (e.g., detects or receives) an ECG of an individual. The individual is receiving chest compressions, for instance, such that the ECG includes a chest compression artifact. For example, the ECG is obtained during a CPR period during which the chest compressions are administered to the individual without a pause. In various examples, the medical device detects the ECG based on one or more relative voltages of detection electrodes in contact with the skin of the individual. The relative voltages of the detection electrodes change, for instance, based on the electrical activity of the individual's heart.

At 504, the medical device removes a chest compression artifact from the ECG. In some examples, the medical device removes at least a portion of the chest compression artifact by applying a comb filter, a Kalman filter, an FIR filter, a high-pass filter, a band-reject filter, or any combination thereof. In some cases, the medical device detects the chest compressions applied to the individual during the time period at which the ECG of the individual was detected.

At 506, the medical device identifies motion of the individual. The motion detected at 506 includes irregular motion, in various examples. The motion is detected by one or more motion sensors and corresponds to motion of the individual and/or a surface in contact with the individual (such as the bed of a transport vehicle in which the individual is being transported, a cot on which the individual is disposed, or the like). The motion sensor(s) include, for instance, an accelerometer, a camera, a location tracker (e.g., a GPS device), a gyroscope, a compression detector, a pressure sensor, a speedometer (e.g., of a transport vehicle), or any combination thereof. The motion sensor(s) is integrated into a patch device in contact with (e.g., adhered to) the individual, the medical device itself, a cot supporting the individual, a transport vehicle transporting the individual, a wearable device worn by the individual or a rescuer, a mobile device of an individual or a rescuer, an unmanned vehicle in the vicinity of the individual, or a combination thereof.

In various examples, the medical device distinguishes the irregular motion from motion corresponding to chest compressions administered to the individual. For example, the medical device determines that any motion that is not time-aligned with the chest compressions is irregular motion. In some cases, the medical device determines that any motion corresponding to frequencies outside of a fundamental frequency or harmonics of the chest compressions is irregular motion. In some cases, the medical device determines that any non-periodic detected motion is irregular motion.

At 508, the medical device determines whether a magnitude of the irregular motion is less than a threshold. The irregular motion is defined as a speed, a velocity, an acceleration, a jerk, or any higher-order differential of the position of the individual and/or of the motion sensor(s). The magnitude of the irregular motion is therefore a magnitude of the speed, the velocity, the acceleration, the jerk, or any other higher-order differential of the position of the individual and/or of the motion sensor(s). In some examples, the threshold is predetermined. For example, the threshold corresponds to an experimentally derived motion value that corresponds to an unacceptable level of certainty with whatever technique is used to filter the chest compression artifact from the ECG and/or to generate a recommendation based on the filtered ECG. In some cases, the medical device compares the magnitudes of multiple types of motion (e.g., the velocity and acceleration) to multiple respective thresholds, and determines whether the magnitudes of the multiple types of motion are less than their respective thresholds.

If the medical device determines that the magnitude of the irregular motion is less than the threshold, then the process 500 proceeds to 512. At 512, the medical device outputs a shock recommendation. For example, the medical device generates the shock recommendation based on the filtered ECG. In some examples, the medical device generates a shock index based on the filtered ECG, compares the shock index to one or more thresholds, and generates a shock recommendation based on the comparison of the shock index to the threshold(s). The shock recommendation recommends that the individual be administered a defibrillation shock, that no defibrillation shock should be administered to the individual, or an indeterminate decision. In various manual mode implementations, the user decides whether to administer the defibrillation shock, or whether to pause CPR for additional analysis, based on the shock recommendation.

If, on the other hand, the medical device determines that the magnitude of the irregular motion is greater than or equal to the threshold, then the process 500 proceeds to 510. At 510, the medical device outputs an accuracy of the shock recommendation. In some examples, the medical device determines the accuracy based on the shock index. For example, the shock index corresponds to a certainty or probability that a shockable rhythm (e.g., VF or pulseless V-Tach) is present in the filtered ECG. In some cases, the medical device determines the accuracy based on the irregular motion detected at 506. For example, the irregular motion is negatively correlated with the accuracy of the shock recommendation. The process 500 also proceeds to 512, such that the accuracy is output with the shock recommendation.

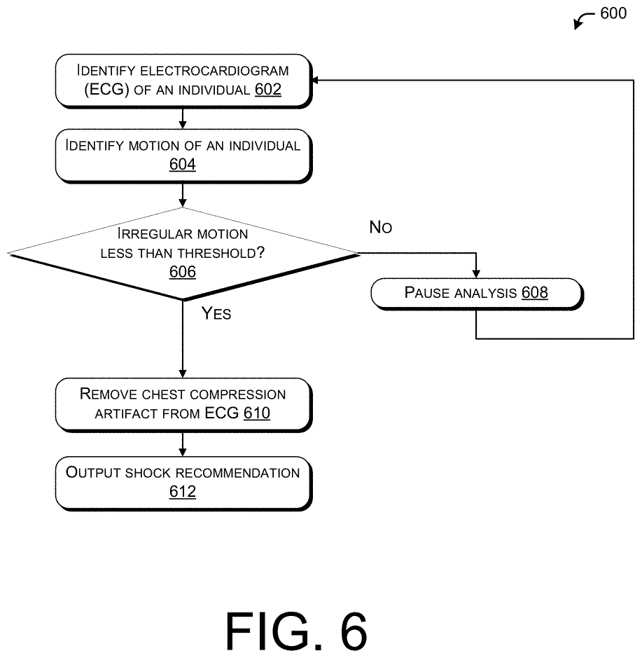

FIG. 6 illustrates an example process 600 for selectively pausing an advisory mode of a defibrillator based on irregular motion detection. In various cases, the process 600 is performed by a medical device, such as the monitor-defibrillator 106 described above with reference to FIG. 1 and/or the defibrillator 300 described above with reference to FIG. 3.

At 602, the medical device identifies (e.g., detects or receives) an ECG of an individual. The individual is receiving chest compressions, for instance, such that the ECG includes a chest compression artifact. For example, the ECG is obtained during a CPR period during which the chest compressions are administered to the individual without a pause. In various examples, the medical device detects the ECG based on one or more relative voltages of detection electrodes in contact with the skin of the individual. The relative voltages of the detection electrodes change, for instance, based on the electrical activity of the individual's heart.

At 604, the medical device identifies motion of the individual. The motion detected at 604 includes irregular motion, in various examples. The motion is detected by one or more motion sensors and corresponds to motion of the individual and/or a surface in contact with the individual (such as the bed of a transport vehicle in which the individual is being transported, a cot on which the individual is disposed, or the like). The motion sensor(s) include, for instance, an accelerometer, a camera, a location tracker (e.g., a GPS device), a gyroscope, a compression detector, a pressure sensor, a speedometer (e.g., of a transport vehicle), or any combination thereof. The motion sensor(s) is integrated into a patch device in contact with (e.g., adhered to) the individual, the medical device itself, a cot supporting the individual, a transport vehicle transporting the individual, a wearable device worn by the individual or a rescuer, a mobile device of an individual or a rescuer, an unmanned vehicle in the vicinity of the individual, or a combination thereof.

In various examples, the medical device distinguishes the irregular motion from motion corresponding to chest compressions administered to the individual. For example, the medical device determines that any motion that is not time-aligned with the chest compressions is irregular motion. In some cases, the medical device determines that any motion corresponding to frequencies outside of a fundamental frequency or harmonics of the chest compressions is irregular motion. In some cases, the medical device determines that any non-periodic detected motion is irregular motion.

At 606, the medical device determines whether a magnitude of the irregular motion is less than a threshold. The irregular motion is defined as a speed, a velocity, an acceleration, a jerk, or any higher-order differential of the position of the individual and/or of the motion sensor(s). The magnitude of the irregular motion is therefore a magnitude of the speed, the velocity, the acceleration, the jerk, or any other higher-order differential of the position of the individual and/or of the motion sensor(s). In some examples, the threshold is predetermined. For example, the threshold corresponds to an experimentally derived motion value that corresponds to an unacceptable level of certainty with whatever technique is used to filter the chest compression artifact from the ECG and/or to generate a recommendation based on the filtered ECG. In some cases, the medical device compares the magnitudes of multiple types of motion (e.g., the velocity and acceleration) to multiple respective thresholds, and determines whether the magnitudes of the multiple types of motion are less than their respective thresholds.

If the medical device determines that the magnitude of the irregular motion is greater than or equal to the threshold, then the process 600 proceeds to 608. At 608, the medical device at least temporarily pauses analysis of the ECG. For instance, the medical device refrains from removing the chest compression artifact from the ECG or otherwise filtering the ECG. The medical device refrains from generating or outputting a shock recommendation at 608, for instance.

If, on the other hand, the medical device determines that the magnitude of the irregular motion is less than the threshold, the process 600 proceeds to 610. At 610, the medical device removes the chest compression artifact from the ECG. In some examples, the medical device removes at least a portion of the chest compression artifact by applying a comb filter, a Kalman filter, an FIR filter, a high-pass filter, a band-reject filter, or any combination thereof.

Once the chest compression artifact is removed from the ECG, the medical device outputs a shock recommendation at 612. For example, the medical device generates the shock recommendation based on the filtered ECG. In some examples, the medical device generates a shock index based on the filtered ECG, compares the shock index to one or more thresholds, and generates a shock recommendation based on the comparison of the shock index to the threshold(s). The shock recommendation recommends that the individual be administered a defibrillation shock, that no defibrillation shock should be administered to the individual, or an indeterminate decision. In various manual mode implementations, the user decides whether to administer the defibrillation shock, or whether to pause CPR for additional analysis, based on the shock recommendation.

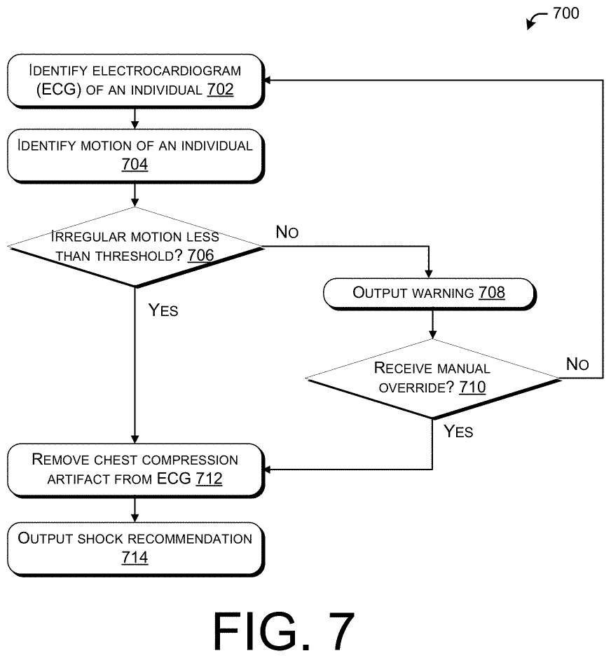

FIG. 7 illustrates an example process 700 for selectively allowing a user to manually override pausing of an advisory mode of a defibrillator based on irregular motion detection. In various cases, the process 700 is performed by a medical device, such as the monitor-defibrillator 106 described above with reference to FIG. 1 and/or the defibrillator 300 described above with reference to FIG. 3.

At 702, the medical device identifies (e.g., detects or receives) an ECG of an individual. The individual is receiving chest compressions, for instance, such that the ECG includes a chest compression artifact. For example, the ECG is obtained during a CPR period during which the chest compressions are administered to the individual without a pause. In various examples, the medical device detects the ECG based on one or more relative voltages of detection electrodes in contact with the skin of the individual. The relative voltages of the detection electrodes change, for instance, based on the electrical activity of the individual's heart.

At 704, the medical device identifies motion of the individual. The motion detected at 704 includes irregular motion, in various examples. The motion is detected by one or more motion sensors and corresponds to motion of the individual and/or a surface in contact with the individual (such as the bed of a transport vehicle in which the individual is being transported, a cot on which the individual is disposed, or the like). The motion sensor(s) include, for instance, an accelerometer, a camera, a location tracker (e.g., a GPS device), a gyroscope, a compression detector, a pressure sensor, a speedometer (e.g., of a transport vehicle), or any combination thereof. The motion sensor(s) is integrated into a patch device in contact with (e.g., adhered to) the individual, the medical device itself, a cot supporting the individual, a transport vehicle transporting the individual, a wearable device worn by the individual or a rescuer, a mobile device of an individual or a rescuer, an unmanned vehicle in the vicinity of the individual, or a combination thereof.

In various examples, the medical device distinguishes the irregular motion from motion corresponding to chest compressions administered to the individual. For example, the medical device determines that any motion that is not time-aligned with the chest compressions is irregular motion. In some cases, the medical device determines that any motion corresponding to frequencies outside of a fundamental frequency or harmonics of the chest compressions is irregular motion. In some cases, the medical device determines that any non-periodic detected motion is irregular motion.

At 706, the medical device determines whether a magnitude of the irregular motion is less than a threshold. The irregular motion is defined as a speed, a velocity, an acceleration, a jerk, or any higher-order differential of the position of the individual and/or of the motion sensor(s). The magnitude of the irregular motion is therefore a magnitude of the speed, the velocity, the acceleration, the jerk, or any other higher-order differential of the position of the individual and/or of the motion sensor(s). In some examples, the threshold is predetermined. For example, the threshold corresponds to an experimentally derived motion value that corresponds to an unacceptable level of certainty with whatever technique is used to filter the chest compression artifact from the ECG and/or to generate a recommendation based on the filtered ECG. In some cases, the medical device compares the magnitudes of multiple types of motion (e.g., the velocity and acceleration) to multiple respective thresholds, and determines whether the magnitudes of the multiple types of motion are less than their respective thresholds.

If the medical device determines that the magnitude of the irregular motion is greater than or equal to the threshold, then the process 700 proceeds to 708. At 708, the medical device outputs a warning. For example, the medical device outputs the warning visually on a display of the medical device, audibly by a speaker of the medical device, haptically as vibration of at least a portion of the medical device, or the like. The warning, for instance, indicates that a shock recommendation is potentially inaccurate.

At 710, the medical device determines whether a manual override is received. In various implementations, the warning is output with an option for a manual override. In some examples, the medical device is configured to receive an input signal from a user based on the warning. For instance, the medical device includes a touchscreen and a touch sensor overlapping a GUI element that includes the warning receives the input signal. The input signal corresponds to the manual override.

If the medical device does not receive the manual override, then the process 700 returns to 702. However, if the medical device receives the manual override, the process 700 proceeds to 712. Similarly, if the irregular motion is determined to be less than the threshold at 706, then the process 700 arrives at 712. At 712, the medical device removes the chest compression artifact from the ECG. In some examples, the medical device removes at least a portion of the chest compression artifact by applying a comb filter, a Kalman filter, an FIR filter, a high-pass filter, a band-reject filter, or any combination thereof.

Once the chest compression artifact is removed from the ECG, the medical device outputs a shock recommendation at 714. For example, the medical device generates the shock recommendation based on the filtered ECG. In some examples, the medical device generates a shock index based on the filtered ECG, compares the shock index to one or more thresholds, and generates a shock recommendation based on the comparison of the shock index to the threshold(s). The shock recommendation recommends that the individual be administered a defibrillation shock, that no defibrillation shock should be administered to the individual, or an indeterminate decision. In various manual mode implementations, the user decides whether to administer the defibrillation shock, or whether to pause CPR for additional analysis, based on the shock recommendation.

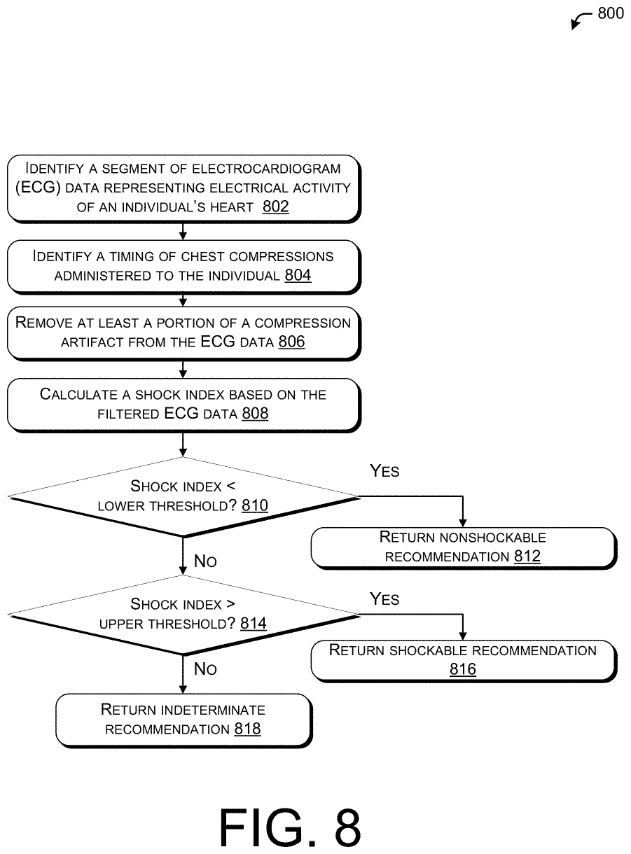

FIG. 8 illustrates an example process 800 for identifying a shockable rhythm in ECG data that includes a chest compression artifact. The process 800 is performed by a medical device, such as the monitor-defibrillator 106 described above with reference to FIG. 1 and/or the defibrillator 300 described above with reference to FIG. 3.

At 802, the medical device identifies a segment of ECG data representing an electrical activity of an individual's heart when the individual is receiving chest compressions. The ECG data is obtained by detecting one or more relative voltages between electrodes connected to the chest of the individual, for instance. The ECG data is digital data representing the detected voltages, for example. According to various implementations, the chest compressions generate noise in the ECG data. The noise is at least partly based on jostling or movement of the electrodes on the skin of the individual, for example. An artifact is present in the ECG data based on the chest compressions. If the raw ECG data is output to a user, the chest compression artifact makes the ECG data difficult for the user to evaluate, in some cases. For instance, the user may have difficulty manually discerning whether a shockable rhythm (e.g., VF or pulseless V-Tach) is present in the ECG data. Accordingly, the medical device removes the artifact and automatically determines whether the shockable rhythm is present.

The segment is selected from the ECG data. As used herein, the term “segment” can refer to a subset of data that are obtained from a first time to a second time, wherein the first time occurs after the time of the first datapoint in the data and/or the second time occurs before the time of the last datapoint in the data. In some cases, the data in the segment are obtained over a time interval. The time interval, for example, is at least a minimum period and no longer than a maximum period. The minimum period, for instance, is 3 seconds, 4 seconds, 8 seconds, 10 seconds, or another time interval. The maximum period, for example, is 12 seconds, 20 seconds, 30 seconds, or some other time interval.

At 804, the medical device identifies chest compressions administered to the individual. In some cases, the medical device determines when the chest compressions are administered based on a signal from a chest compression monitor, which in some cases is disposed on the chest of the individual includes at least one accelerometer and/or gyroscope that detects chest compressions administered to the individual. In some examples, the medical device detects an electrical impedance between two or more electrodes in contact with the individual and determines when the chest compressions are administered based on the electrical impedance. The chest compressions are administered to the individual during a time period at which the segment of the ECG data is detected, such that the chest compressions cause the chest compression artifact.

At 806, the medical device generates filtered ECG data by removing the chest compression artifact of the selected segment of the ECG data. The chest compression artifact has a fundamental that is between 1.5 to 2 Hz, in various examples. However, heart rhythm features (e.g., a VF rhythm, a V-tach rhythm, QRS complexes, and other inherent heart rhythms) are typically defined by higher frequencies. In some examples, the medical device applies a filter to the detected ECG segment, such as an adaptive filter (e.g., a Wiener filter, a Kalman filter, or the like), an nth order filter (e.g., a zero-th order filter) a comb filter, an inverse comb filter, a high-pass filter, a band reject filter, a finite impulse response (FIR) filter, an infinite impulse response (IIR) filter, or a combination thereof. In some cases, the medical device converts the ECG segment from the time domain into the frequency (e.g., a Fourier) domain, a Laplace domain, a Z-transform domain, or a wavelet (e.g., a continuous wavelet transform, a discrete wavelet transform, etc.) domain, and removes at least a portion of the chest compression artifact by processing the converted ECG. According to some examples, the medical device identifies and subtracts the chest compression artifact. For instance, the medical device identifies and subtracts the chest compression artifact based on the detected chest compressions. For example, the medical device cross-correlates the ECG segment with data corresponding to the chest compressions (e.g., the impedance, the acceleration of the compression detector, the velocity of the compression detector, etc.), identifies the chest compression artifact based on the cross-correlation, and subtracts the chest compression artifact from the ECG segment. In some instances, the medical device denoises the ECG segment. For example, the medical device removes at least a portion of the chest compression artifact by performing spectral subtraction on the ECG segment.

Optionally, the medical device applies additional filtering techniques to reduce the harmonics of the chest compression artifact in the selected segment of the ECG data. For example, the medical device applies a comb filter with multiple stopbands that correspond to the fundamental frequency of the chest compressions administered to the individual and one or more harmonics of the fundamental frequency.

At 808, the medical device calculates a shock index based on the filtered ECG data. The shock index, for example, corresponds to a likelihood that the original ECG data and/or the filtered ECG data exhibits a rhythm that is treatable with defibrillation. For example, the shock index relates to the likelihood that the filtered ECG data is indicative that the individual is exhibiting VF or pulseless V-Tach. In some examples, the medical device calculates the shock index by detecting a shockable rhythm (e.g., VF or pulseless V-Tach) in the filtered ECG data. In some cases, the medical device performs a rules-based analysis on the filtered ECG data. In some examples, the shock index is generated based on an amplitude magnitude spectrum area (AMSA) of the filtered ECG data, an amplitude of the filtered ECG data, a frequency of the filtered ECG data, or a combination thereof. In some implementations, the medical device calculates the shock index by determining a spectral similarity between the filtered ECG and a sample ECG with a known shockable rhythm (e.g., VF or pulseless V-Tach) and/or by determining a spectral dissimilarity between the filtered ECG and a sample ECG with a known nonshockable rhythm (e.g., asystole, a sinus rhythm including QRS complexes, etc.). In some examples the medical device uses non-ECG data to generate the shock index, at least in part. For instance, the medical device generates the shock index based on a non-ECG physiological parameter (e.g., a heart rate level or waveform, a temperature level or waveform, an airway CO₂ level or waveform, an oxygenation level or waveform, a blood pressure level or waveform, etc.) of the individual, a type of equipment monitoring the individual, a demographic of the individual, or a combination thereof. In some examples, the shock index is calculated based on a regression (e.g., linear regression, binary regression, polynomial regression, logistic regression, nonlinear regression, nonparametric regression, etc.) model outputting a probability that the filtered ECG exhibits a shockable rhythm based on one or more characteristics of the filtered ECG. In various implementations, the medical device generates the shock index based on one or more analysis factors.

At 810, the medical device determines whether the shock index is less than a lower threshold. The lower threshold is selected, for instance, based on an acceptable level of uncertainty regarding a nonshockable recommendation. In some cases, the lower threshold is user-selected, such that the lower threshold is calculated based on an input signal from a user. In some cases, the lower threshold is determined based on one or more analysis factors. If the medical device determines that the shock index is less than the lower threshold, the medical device returns a nonshockable recommendation at 812.

If, on the other hand, the medical device determines that the shock index is greater than or equal to the lower threshold, the process 800 proceeds to 814. At 814, the medical device determines whether the shock index is greater than the upper threshold. The upper threshold is selected, for instance, based on an acceptable level of uncertainty regarding a shockable recommendation. In some cases, the upper threshold is user-selected, such that the upper threshold is calculated based on an input signal from a user. In some examples, the upper threshold is determined based on one or more analysis factors. If the medical device determines that the shock index is greater than the upper threshold, the medical device returns a shockable recommendation at 816.

However, if the medical device determines that the shock index is less than or equal to the upper threshold, then the medical device returns an indeterminate recommendation at 818. The indeterminate decision means that the medical device is unable to conclude whether the shockable rhythm is present with a sufficient level of certainty. The level of certainty, in some cases, is predetermined and/or selected by a user.

In various cases, the medical device performs the process 800 repeatedly, periodically, or a combination thereof. For example, upon returning a recommendation, the medical device repeats the process 800 by identifying another segment of ECG data. In some cases, the medical device initiates the process 800 (e.g., begins 802) at a particular frequency, such that the medical device may be performing the process 800 multiple times, in parallel, at a time. If the medical device determines multiple recommendations based on repeatedly and/or periodically performing the process 800, the medical device outputs (e.g., to the user) a recommendation based on the most recently returned shock decision.

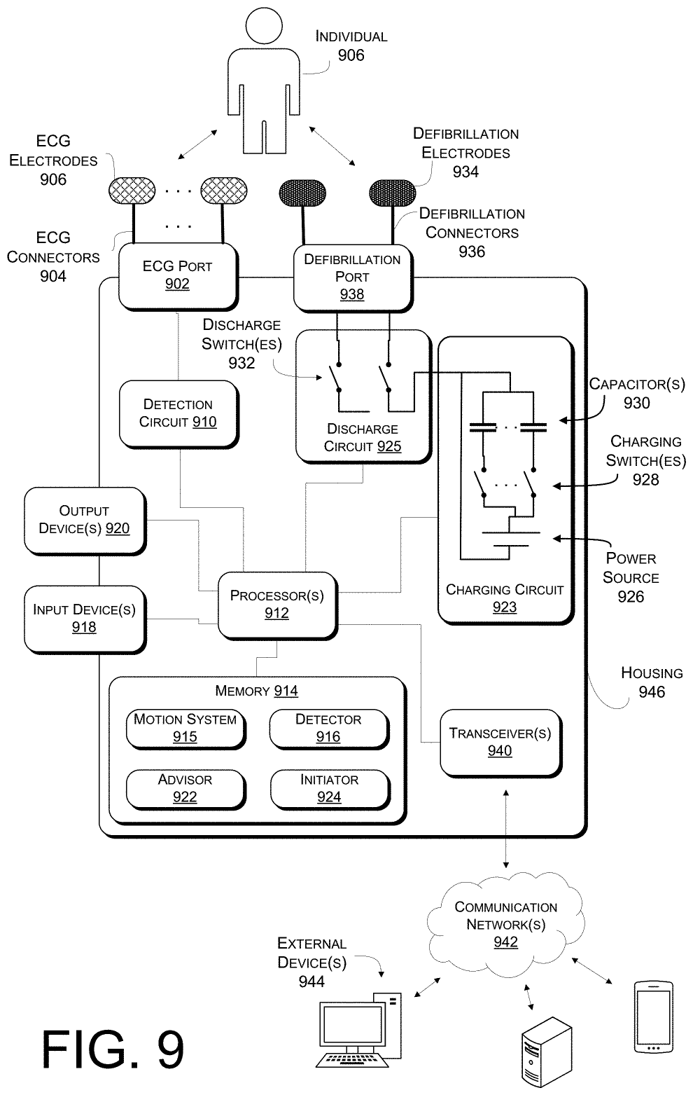

FIG. 9 illustrates an example of an external defibrillator 900 configured to perform various functions described herein. For example, the external defibrillator 900 is the monitor-defibrillator 106 described above with reference to FIG. 1 and/or the defibrillator 300 described above with reference to FIG. 3.

The external defibrillator 900 includes an electrocardiogram (ECG) port 902 connected to multiple ECG connectors 904. In some cases, the ECG connectors 904 are removeable from the ECG port 902. For instance, the ECG connectors 904 are plugged into the ECG port 902. The ECG connectors 904 are connected to ECG electrodes 906, respectively. In various implementations, the ECG electrodes 906 are disposed on different locations on an individual 908. A detection circuit 910 is configured to detect relative voltages between the ECG electrodes 906. These voltages are indicative of the electrical activity of the heart of the individual 908.

In various implementations, the ECG electrodes 906 are in contact with the different locations on the skin of the individual 908. In some examples, a first one of the ECG electrodes 906 is placed on the skin between the heart and right arm of the individual 908, a second one of the ECG electrodes 906 is placed on the skin between the heart and left arm of the individual 908, and a third one of the ECG electrodes 906 is placed on the skin between the heart and a leg (either the left leg or the right leg) of the individual 908. In these examples, the detection circuit 908 is configured to measure the relative voltages between the first, second, and third ECG electrodes 906. Respective pairings of the ECG electrodes 906 are referred to as “leads,” and the voltages between the pairs of ECG electrodes 906 are known as “lead voltages.” In some examples, more than three ECG electrodes 906 are included, such that 5-lead or 12-lead ECG signals are detected by the detection circuit 910.

The detection circuit 910 includes at least one analog circuit, at least one digital circuit, or a combination thereof. The detection circuit 910 receives the analog electrical signals from the ECG electrodes 906, via the ECG port 902 and the ECG connectors 904. In some cases, the detection circuit 910 includes one or more analog filters configured to filter noise and/or artifact from the electrical signals. The detection circuit 910 includes an analog-to-digital (ADC) in various examples. The detection circuit 910 generates a digital signal indicative of the analog electrical signals from the ECG electrodes 906. This digital signal can be referred to as an “ECG signal” or an “ECG.”

In some cases, the detection circuit 910 further detects an electrical impedance between at least one pair of the ECG electrodes 906. For example, the detection circuit 910 includes, or otherwise controls, a power source that applies a known voltage across a pair of the ECG electrodes 906 and detects a resultant current between the pair of the ECG electrodes 906. The impedance is generated based on the applied voltage and the resultant current. In various cases, the impedance corresponds to respiration of the individual 908, chest compressions performed on the individual 908, and other physiological states of the individual 908. In various examples, the detection circuit 910 includes one or more analog filters configured to filter noise and/or artifact from the resultant current. The detection circuit 910 generates a digital signal indicative of the impedance using an ADC. This digital signal can be referred to as an “impedance signal” or an “impedance.”

The detection circuit 910 provides the ECG signal and/or the impedance signal one or more processors 912 in the external defibrillator 900. In some implementations, the processor(s) 912 includes a central processing unit (CPU), a graphics processing unit (GPU), both CPU and GPU, or other processing unit or component known in the art.

The processor(s) 912 is operably connected to memory 914. In various implementations, the memory 912 is volatile (such as random access memory (RAM)), non-volatile (such as read only memory (ROM), flash memory, etc.) or some combination of the two. The memory 914 stores instructions that, when executed by the processor(s) 912, causes the processor(s) 912 to perform various operations. In various examples, the memory 914 stores methods, threads, processes, applications, objects, modules, any other sort of executable instruction, or a combination thereof. In some cases, the memory 914 stores files, databases, or a combination thereof. In some examples, the memory 914 includes, but is not limited to, RAM, ROM, electrically erasable programmable read-only memory (EEPROM), flash memory, or any other memory technology. In some examples, the memory 914 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 912 and/or the external defibrillator 900. In some cases, the memory 914 at least temporarily stores the ECG signal and/or the impedance signal.

In particular implementations, the memory 914 includes instructions that, when executed by the processor(s) 912, cause the processor(s) to perform operations of a motion system 915. The motion system 915 causes the processor(s) 912 to detect irregular motion of the individual 906 based on signals received by one or more motion sensors. In some cases, the motion system 915 causes the processor(s) 912 to refrain from filtering the ECG signal, to refrain from outputting a shock recommendation, to output a certainty of the shock recommendation, to selectively filter and/or output a shock recommendation in response to receiving a manual override signal, or a combination thereof, when the processor(s) 912 determine that the irregular motion is greater than or equal to a threshold.

In various examples, the memory 914 includes a detector 916, which causes the processor(s) 912 to determine, based on the ECG signal and/or the impedance signal, whether the individual 908 is exhibiting a particular heart rhythm. For instance, the processor(s) 912 determines whether the individual 908 is experiencing a shockable rhythm that is treatable by defibrillation. Examples of shockable rhythms include ventricular fibrillation (VF) and pulseless ventricular tachycardia (V-Tach). In some examples, the processor(s) 912 determines whether any of a variety of different rhythms (e.g., asystole, sinus rhythm, atrial fibrillation (AF), etc.) are present in the ECG signal.

The processor(s) 912 is operably connected to one or more input devices 918 and one or more output devices 920. Collectively, the input device(s) 918 and the output device(s) 920 function as an interface between a user and the defibrillator 900. The input device(s) 918 is configured to receive an input from a user and includes at least one of a keypad, a cursor control, a touch-sensitive display (e.g., a touchscreen), a voice input device (e.g., a speaker), a haptic feedback device, or any combination thereof. In some examples, the input device(s) 918 include one or more motion sensors, such as an accelerometer, a camera, a gyroscope, a speedometer, a compression detector, a location tracker, or any combination thereof. The output device(s) 920 includes at least one of a display, a speaker, a haptic output device, a printer, or any combination thereof. In various examples, the processor(s) 912 causes a display among the input device(s) 918 to visually output a waveform of the ECG signal and/or the impedance signal. In some implementations, the input device(s) 918 includes one or more touch sensors, the output device(s) 920 includes a display screen, and the touch sensor(s) are integrated with the display screen. Thus, in some cases, the external defibrillator 900 includes a touchscreen configured to receive user input signal(s) and visually output physiological parameters, such as the ECG signal and/or the impedance signal.

In some examples, the memory 914 includes an advisor 922, which, when executed by the processor(s) 912, causes the processor(s) 912 to generate advice and/or control the output device(s) 920 to output the advice to a user (e.g., a rescuer). In some examples, the processor(s) 912 provides, or causes the output device(s) 920 to provide, an instruction to perform CPR on the individual 908. In some cases, the processor(s) 912 evaluates, based on the ECG signal, the impedance signal, or other physiological parameters, CPR being performed on the individual 908 and causes the output device(s) 920 to provide feedback about the CPR in the instruction. According to some examples, the processor(s) 912, upon identifying that a shockable rhythm is present in the ECG signal, causes the output device(s) 920 to output an instruction and/or recommendation to administer a defibrillation shock to the individual 908.

The memory 914 also includes an initiator 924 which, when executed by the processor(s) 912, causes the processor(s) 912 to control other elements of the external defibrillator 900 in order to administer a defibrillation shock to the individual 908. In some examples, the processor(s) 912 executing the initiator 924 selectively causes the administration of the defibrillation shock based on determining that the individual 908 is exhibiting the shockable rhythm and/or based on an input from a user (received, e.g., by the input device(s) 918. In some cases, the processor(s) 912 causes the defibrillation shock to be output at a particular time, which is determined by the processor(s) 912 based on the ECG signal and/or the impedance signal.

The processor(s) 912 is operably connected to a charging circuit 923 and a discharge circuit 925. In various implementations, the charging circuit 923 includes a power source 926, one or more charging switches 928, and one or more capacitors 930. The power source 926 includes, for instance, a battery. The processor(s) 912 initiates a defibrillation shock by causing the power source 926 to charge at least one capacitor among the capacitor(s) 930. For example, the processor(s) 912 activates at least one of the charging switch(es) 928 in the charging circuit 923 to complete a first circuit connecting the power source 926 and the capacitor to be charged. Then, the processor(s) 912 causes the discharge circuit 925 to discharge energy stored in the charged capacitor across a pair of defibrillation electrodes 930, which are in contact with the individual 908. For example, the processor(s) 912 deactivates the charging switch(es) 928 completing the first circuit between the capacitor(s) 930 and the power source 926, and activates one or more discharge switches 932 completing a second circuit connecting the charged capacitor 930 and at least a portion of the individual 908 disposed between defibrillation electrodes 934. Although not illustrated in FIG. 9, in some implementations, the discharge circuit 925 includes an H-bridge over which the energy from the capacitor(s) 930 is discharged across the defibrillation electrodes 930.

The energy is discharged from the defibrillation electrodes 934 in the form of a defibrillation shock. For example, the defibrillation electrodes 934 are connected to the skin of the individual 908 and located at positions on different sides of the heart of the individual 908, such that the defibrillation shock is applied across the heart of the individual 908. The defibrillation shock, in various examples, depolarizes a significant number of heart cells in a short amount of time. The defibrillation shock, for example, interrupts the propagation of the shockable rhythm (e.g., VF or pulseless V-Tach) through the heart. In some examples, the defibrillation shock is 200 J or greater with a duration of about 0.015 seconds. In some cases, the defibrillation shock has a multiphasic (e.g., biphasic) waveform. The discharge switch(es) 932 are controlled by the processor(s) 912, for example. In various implementations, the defibrillation electrodes 934 are connected to defibrillation connectors 936. The defibrillation connectors 936 are connected to a defibrillation port 938, in implementations. According to various examples, the defibrillation connectors 936 are removable from the defibrillation port 938. For example, the defibrillation connectors 936 are plugged into the defibrillation port 938.

In various implementations, the processor(s) 912 is operably connected to one or more transceivers 940 that transmit and/or receive data over one or more communication networks 942. For example, the transceiver(s) 940 includes a network interface card (NIC), a network adapter, a local area network (LAN) adapter, or a physical, virtual, or logical address to connect to the various external devices and/or systems. In various examples, the transceiver(s) 940 includes any sort of wireless transceivers capable of engaging in wireless communication (e.g., radio frequency (RF) communication). For example, the communication network(s) 942 includes one or more wireless networks that include a 3^{rd} Generation Partnership Project (3GPP) network, such as a Long Term Evolution (LTE) radio access network (RAN) (e.g., over one or more LE bands), a New Radio (NR) RAN (e.g., over one or more NR bands), or a combination thereof. In some cases, the transceiver(s) 940 includes other wireless modems, such as a modem for engaging in WI-FI®, WIGIG®, WIMAX®, BLUETOOTH®, or infrared communication over the communication network(s) 942.

The defibrillator 900 is configured to transmit and/or receive data (e.g., ECG data, impedance data, data indicative of one or more detected heart rhythms of the individual 908, data indicative of one or more defibrillation shocks administered to the individual 908, etc.) with one or more external devices 944 via the communication network(s) 942. The external devices 944 include, for instance, mobile devices (e.g., mobile phones, smart watches, etc.), Internet of Things (loT) devices, medical devices, computers (e.g., laptop devices, servers, etc.), motion sensors, transport vehicles, cots, unmanned vehicles, wearable devices, or any other type of computing device configured to communicate over the communication network(s) 942. In some examples, the external device(s) 944 is located remotely from the defibrillator 900, such as at a remote clinical environment (e.g., a hospital). According to various implementations, the processor(s) 912 causes the transceiver(s) 940 to transmit data to the external device(s) 944. In some cases, the transceiver(s) 940 receives data from the external device(s) 944 and the transceiver(s) 940 provide the received data to the processor(s) 912 for further analysis.

In various implementations, the external defibrillator 900 also includes a housing 946 that at least partially encloses other elements of the external defibrillator 900. For example, the housing 946 encloses the detection circuit 910, the processor(s) 912, the memory 914, the charging circuit 923, the transceiver(s) 940, or any combination thereof. In some cases, the input device(s) 918 and output device(s) 920 extend from an interior space at least partially surrounded by the housing 946 through a wall of the housing 946. In various examples, the housing 946 acts as a barrier to moisture, electrical interference, and/or dust, thereby protecting various components in the external defibrillator 900 from damage.

In some implementations, the external defibrillator 900 is an automated external defibrillator (AED) operated by an untrained user (e.g., a bystander, layperson, etc.) and can be operated in an automatic mode. In automatic mode, the processor(s) 912 automatically identifies a rhythm in the ECG signal, makes a decision whether to administer a defibrillation shock, charges the capacitor(s) 930, discharges the capacitor(s) 930, or any combination thereof. In some cases, the processor(s) 912 controls the output device(s) 920 to output (e.g., display) a simplified user interface to the untrained user. For example, the processor(s) 912 refrains from causing the output device(s) 920 to display a waveform of the ECG signal and/or the impedance signal to the untrained user, in order to simplify operation of the external defibrillator 900.

In some examples, the external defibrillator 900 is a monitor-defibrillator utilized by a trained user (e.g., a clinician, an emergency responder, etc.) and can be operated in a manual mode or the automatic mode. When the external defibrillator 900 operates in manual mode, the processor(s) 912 cause the output device(s) 920 to display a variety of information that may be relevant to the trained user, such as waveforms indicating the ECG data and/or impedance data, notifications about detected heart rhythms, and the like.

### EXAMPLE CLAUSES

1. A defibrillation system, including: a motion detector identifying a motion of an individual receiving chest compressions; detection electrodes contacting the skin of the individual; a detection circuit detecting an electrocardiogram (ECG) of the individual based on a relative voltage between the detection electrodes; an output device; a processor; and memory storing instructions that, when executed by the processor, cause the processor to perform operations including: determining that the motion of the individual is lower than a threshold; based on determining that the motion of the individual is lower than the threshold, generating a filtered ECG by removing, from the ECG, an artifact corresponding to the chest compressions; determining that a shockable rhythm is present in the filtered ECG; and based on determining that the shockable rhythm is present in the filtered ECG, causing the output device to output a recommendation to administer a defibrillation shock to the individual.
2. The defibrillation system of clause 1, wherein the motion detector includes an accelerometer, a camera, a speedometer, a location device, or a location sensor.
3. The defibrillation system of clause 1 or 2, wherein the motion includes irregular motion that is independent of the chest compressions received by the individual.
4. A medical device, including: a motion sensor configured to identify a motion of an individual receiving chest compressions; a processor; and memory storing instructions that, when executed by the processor, cause the processor to perform operations including: determining that the motion of the individual is lower than a threshold; identifying an electrocardiogram (ECG) of the individual; based on determining that the motion of the individual is lower than the threshold, generating a filtered ECG by removing, from the ECG, an artifact corresponding to the chest compressions; determining that a shockable rhythm is present in the filtered ECG; and based on determining that the shockable rhythm is present in the filtered ECG, outputting a recommendation to administer a defibrillation shock to the individual.
5. The medical device of clause 4, wherein the motion includes irregular motion.
6. The medical device of clause 5, wherein the irregular motion is independent of the chest compressions received by the individual.
7. The medical device of any one of clauses 4 to 6, wherein the motion sensor includes an accelerometer, a camera, a speedometer, a navigation device, or a location sensor.
8. The medical device of any one of clauses 4 to 7, further including: multiple motion sensors including the motion sensor, wherein the motion sensors are physically connected to one or more of the individual, to a cot supporting the individual, to a vehicle transporting the individual, to a mobile device, to a wearable device, or to an unmanned aerial vehicle (UAV).
9. The medical device of any one of clauses 4 to 8, wherein the shockable rhythm includes ventricular fibrillation or pulseless ventricular tachycardia.
10. The medical device of any one of clauses 4 to 9, further including: a display configured to output a visual signal indicative of the recommendation to administer the defibrillation shock to the individual; or a speaker configured to output an audible signal indicative of the recommendation to administer the defibrillation shock to the individual.
11. The medical device of any one of clauses 4 to 10, the threshold being a first threshold, wherein determining that the shockable rhythm is present in the filtered ECG includes: generating a shock index based on the filtered ECG; and comparing the shock index to a second threshold.
12. The medical device of any one of clauses 4 to 11, further including: electrodes physically contacting the individual; a discharge circuit selectively outputting the defibrillation shock to the electrodes; and an input device receiving a user input signal, wherein the operations further include: storing, in the memory, data indicative of the filtered ECG, the recommendation, and the defibrillation shock; and causing the discharge circuit to output the defibrillation shock based on the user input signal.
13. A method performed by a medical device, the method including: identifying an irregular motion of an individual; determining that the irregular motion of the individual is lower than a threshold; identifying an electrocardiogram (ECG) of the individual; based on determining that the irregular motion of the individual is lower than the threshold, determining that a shockable rhythm is present in the ECG; and based on determining that the shockable rhythm is present in the ECG, outputting a recommendation to administer a defibrillation shock to the individual.
14. The method of clause 13, wherein the irregular motion is independent of chest compressions received by the individual.
15. The method of clause 13 or 14, wherein identifying the irregular motion of the individual includes: receiving a signal indicative of the irregular motion from a motion sensor, the motion sensor including an accelerometer, a camera, a speedometer of a vehicle transporting the individual, a navigation device, or a location sensor.
16. The method of any one of clauses 13 to 15, wherein the motion sensor is physically connected to the individual, to a cot supporting the individual, to a vehicle transporting the individual, to a mobile device, to a wearable device, or to an unmanned aerial vehicle (UAV).
17. The method of any one of clauses 13 to 16, wherein the shockable rhythm includes ventricular fibrillation or pulseless ventricular tachycardia.
18. The method of any one of clauses 13 to 17, wherein outputting the recommendation to administer a defibrillation shock to the individual includes: outputting a visual signal indicative of the recommendation to administer the defibrillation shock to the individual; or outputting an audible signal indicative of the recommendation to administer the defibrillation shock to the individual.
19. The method of any one of clauses 13 to 18, the threshold being a first threshold, wherein determining that the shockable rhythm is present in the ECG includes: generating a shock index based on the ECG; and comparing the shock index to a second threshold.
20. The method of any one of clauses 13 to 19, further including: receiving an input signal; in response to receiving the input signal, outputting the defibrillation shock to the individual; and storing data indicative of the ECG, the recommendation, and the defibrillation shock.
21. An external defibrillator, including: a motion detector identifying a motion of an individual receiving chest compressions; detection electrodes contacting the skin of the individual; a detection circuit detecting an electrocardiogram (ECG) of the individual based on a relative voltage between the detection electrodes; an output device; a processor; and memory storing instructions that, when executed by the processor, cause the processor to perform operations including: generating a filtered ECG by removing, from the ECG, an artifact corresponding to the chest compressions; determining that a shockable rhythm is present in the filtered ECG; based on determining that the shockable rhythm is present in the filtered ECG, causing the output device to output a recommendation to administer a defibrillation shock to the individual; and determining that the motion of the individual is greater than a threshold.
22. The external defibrillator of clause 21, wherein the operations further include: based on determining that the motion of the individual is greater than the threshold, modifying the recommendation includes causing the output device to output a certainty of the recommendation.
23. The external defibrillator of clause 21 or 22, wherein the motion detector includes an accelerometer, a camera, a speedometer, a navigation device, or a location sensor.
24. The external defibrillator of any one of clauses 21 to 23, wherein the motion includes irregular motion that is independent of the chest compressions received by the individual.
25. A medical device, including: a motion sensor configured to identify a motion of an individual receiving chest compressions; a processor; and memory storing instructions that, when executed by the processor, cause the processor to perform operations including: identifying an electrocardiogram (ECG) of the individual; generating a filtered ECG by removing, from the ECG, an artifact corresponding to the chest compressions; determining whether a shockable rhythm is present in the filtered ECG; based on determining whether the shockable rhythm is present in the filtered ECG, outputting a recommendation of whether to administer a defibrillation shock to the individual; and determining that the motion of the individual is greater than a threshold.
26. The medical device of clause 25, the operations further including: based on determining that the motion of the individual is greater than the threshold: hiding the recommendation; or outputting a confidence level of the recommendation.
27. The medical device of clause 26, further including: a display configured to visually output the recommendation and to visually output the confidence level of the recommendation.
28. The medical device of any one of clauses 25 to 27, wherein the motion of the individual is an irregular motion.
29. The medical device of clause 28, the threshold being a first threshold, wherein the motion sensor is further configured to identify a motion of a cot or transport vehicle supporting the individual, and wherein the operations further include determining whether the motion of the cot or the transport vehicle supporting the individual is greater than a second threshold.
30. The medical device of any one of clauses 25 to 29, wherein the motion sensor includes an accelerometer, a camera, a speedometer, a navigation device, or a location sensor.
31. The medical device of any one of clauses 25 to 30, wherein the motion sensor is physically connected to the individual, to a cot supporting the individual, to a vehicle transporting the individual, to a mobile device, to a wearable device, or to an unmanned aerial vehicle (UAV).
32. The medical device of any one of clauses 25 to 31, wherein the shockable rhythm includes ventricular fibrillation or pulseless ventricular tachycardia.
33. The medical device of any one of clauses 25 to 32, the threshold being a first threshold, wherein determining that the shockable rhythm is present in the filtered ECG includes: generating a shock index based on the filtered ECG; and comparing the shock index to a second threshold.
34. The medical device of clause 33, wherein the operations further include: determining a certainty of the recommendation based on the shock index and the motion; and outputting the certainty of the recommendation.
35. The medical device of any one of clauses 25 to 34, further including: defibrillation electrodes physically contacting the individual; a discharge circuit selectively outputting the defibrillation shock to the defibrillation electrodes; and an input device receiving a user input signal, wherein the operations further include: storing, in the memory, data indicative of the filtered ECG, the recommendation, and the defibrillation shock; and causing the discharge circuit to output the defibrillation shock based on the user input signal.
36. A method performed by a medical device, the method including: identifying an electrocardiogram (ECG) of the individual; based on determining that the motion of the individual is lower than the threshold, generating a filtered ECG by removing, from the ECG, an artifact corresponding to the chest compressions; determining that a shockable rhythm is present in the filtered ECG; generating a recommendation based on the shockable rhythm; identifying a motion associated with an individual receiving chest compressions; determining that the motion associated with the individual is greater than or equal to a threshold; based on determining that the motion associated with the individual is greater than or equal to the threshold, outputting a warning about the recommendation; in response to outputting the warning indicating that the shock recommendation is inaccurate, receiving an input signal corresponding to a manual override; based on determining that the shockable rhythm is present in the filtered ECG and receiving the input signal corresponding to the manual override, outputting a recommendation to administer a defibrillation shock to the individual.
37. The method of clause 36, wherein the motion associated with the individual includes an irregular motion of the individual, a motion of a cot supporting the individual, or a motion of a vehicle transporting the individual, and wherein identifying the motion associated with the individual includes: receiving, from a motion sensor, a signal indicative of the irregular motion, the motion sensor including an accelerometer, a camera, a speedometer of a vehicle transporting the individual, a navigation device, or a location sensor.
38. The method of clause 37, wherein the motion sensor is physically connected to the individual, to the cot supporting the individual, to the vehicle transporting the individual, to a mobile device, to a wearable device, or to an unmanned aerial vehicle (UAV).
39 The method of clause 38, further including: outputting a certainty of the recommendation.
40. The method of clause 39, further including: determining the certainty based on the motion associated with the individual.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

1. A defibrillation system, comprising:
a motion detector identifying a motion of an individual receiving chest compressions;
detection electrodes contacting the skin of the individual;
a detection circuit detecting an electrocardiogram (ECG) of the individual based on a relative voltage between the detection electrodes;
an output device;
a processor; and
memory storing instructions that, when executed by the processor, cause the processor to perform operations comprising:
determining that the motion of the individual is lower than a threshold;
based on determining that the motion of the individual is lower than the threshold, generating a filtered ECG by removing, from the ECG, an artifact corresponding to the chest compressions;
determining that a shockable rhythm is present in the filtered ECG; and
based on determining that the shockable rhythm is present in the filtered ECG, causing the output device to output a recommendation to administer a defibrillation shock to the individual.

2. The defibrillation system of claim 1, wherein the motion detector comprises an accelerometer, a camera, a speedometer, a location device, or a location sensor.

3. The defibrillation system of claim 1, wherein the motion comprises irregular motion that is independent of the chest compressions received by the individual.

4. A medical device, comprising:
a motion sensor configured to identify a motion of an individual receiving chest compressions;
a processor; and
memory storing instructions that, when executed by the processor, cause the processor to perform operations comprising:
determining that the motion of the individual is lower than a threshold;
identifying an electrocardiogram (ECG) of the individual;
based on determining that the motion of the individual is lower than the threshold, generating a filtered ECG by removing, from the ECG, an artifact corresponding to the chest compressions;
determining that a shockable rhythm is present in the filtered ECG; and
based on determining that the shockable rhythm is present in the filtered ECG, outputting a recommendation to administer a defibrillation shock to the individual.

5. The medical device of claim 4, wherein the motion comprises irregular motion.

6. The medical device of claim 5, wherein the irregular motion is independent of the chest compressions received by the individual.

7. The medical device of claim 4, wherein the motion sensor comprises an accelerometer, a camera, a speedometer, a navigation device, or a location sensor.

8. The medical device of claim 4, further comprising: multiple motion sensors comprising the motion sensor, wherein the motion sensors are physically connected to one or more of the individual, to a cot supporting the individual, to a vehicle transporting the individual, to a mobile device, to a wearable device, or to an unmanned aerial vehicle (UAV).

9. The medical device of claim 4, wherein the shockable rhythm comprises ventricular fibrillation or pulseless ventricular tachycardia.

10. The medical device of claim 4, further comprising:
a display configured to output a visual signal indicative of the recommendation to administer the defibrillation shock to the individual; or
a speaker configured to output an audible signal indicative of the recommendation to administer the defibrillation shock to the individual.

11. The medical device of claim 4, the threshold being a first threshold, wherein determining that the shockable rhythm is present in the filtered ECG comprises:
generating a shock index based on the filtered ECG; and
comparing the shock index to a second threshold.

12. The medical device of claim 4, further comprising:
electrodes physically contacting the individual;
a discharge circuit selectively outputting the defibrillation shock to the electrodes; and
an input device receiving a user input signal,
wherein the operations further comprise:
storing, in the memory, data indicative of the filtered ECG, the recommendation, and the defibrillation shock; and
causing the discharge circuit to output the defibrillation shock based on the user input signal.

13. A method performed by a medical device, the method comprising:
identifying an irregular motion of an individual;
determining that the irregular motion of the individual is lower than a threshold;
identifying an electrocardiogram (ECG) of the individual;
based on determining that the irregular motion of the individual is lower than the threshold, determining that a shockable rhythm is present in the ECG; and
based on determining that the shockable rhythm is present in the ECG, outputting a recommendation to administer a defibrillation shock to the individual.

14. The method of claim 13, wherein the irregular motion is independent of chest compressions received by the individual.

15. The method of claim 13, wherein identifying the irregular motion of the individual comprises:
receiving a signal indicative of the irregular motion from a motion sensor, the motion sensor comprising an accelerometer, a camera, a speedometer of a vehicle transporting the individual, a navigation device, or a location sensor.

16. The method of claim 13, wherein the motion sensor is physically connected to the individual, to a cot supporting the individual, to a vehicle transporting the individual, to a mobile device, to a wearable device, or to an unmanned aerial vehicle (UAV).

17. The method of claim 13, wherein the shockable rhythm comprises ventricular fibrillation or pulseless ventricular tachycardia.

18. The method of claim 13, wherein outputting the recommendation to administer a defibrillation shock to the individual comprises:
outputting a visual signal indicative of the recommendation to administer the defibrillation shock to the individual; or
outputting an audible signal indicative of the recommendation to administer the defibrillation shock to the individual.

19. The method of claim 13, the threshold being a first threshold, wherein determining that the shockable rhythm is present in the ECG comprises:
generating a shock index based on the ECG; and
comparing the shock index to a second threshold.

20. The method of claim 13, further comprising:
receiving an input signal;
in response to receiving the input signal, outputting the defibrillation shock to the individual; and
storing data indicative of the ECG, the recommendation, and the defibrillation shock.
